(19) United States
(12) Patent Application Publication
(10) Pub. No.: US 2013/0145488 A1
(43) Pub. Date: Jun. 6, 2013

(54) MESOPOROUS SILICA NANOPARTICLES SUITABLE FOR CO-DELIVERY

(71) Applicants: Kan Wang, Ames, IA (US); Brian G. Trewyn, Golden, CO (US); Susana Martin-Ortigosa, Ames, IA (US); Justin Valenstein, Ames, IA (US)

(72) Inventors: Kan Wang, Ames, IA (US); Brian G. Trewyn, Golden, CO (US); Susana Martin-Ortigosa, Ames, IA (US); Justin Valenstein, Ames, IA (US); Victor Shang-Yi Lin, Ames, IA (US)

(73) Assignee: Iowa State University Research Foundation, Inc., Ames, IA (US)

(21) Appl. No.: 13/707,233

(22) Filed: Dec. 6, 2012

Related U.S. Application Data
(60) Provisional application No. 61/567,477, filed on Dec. 6, 2011.

(51) Int. Cl. C12N 15/89

(52) U.S. Cl. CPC C12N 15/895
USPC 800/21; 435/459; 435/470; 435/471; 435/410; 252/182.12; 800/293

(57) ABSTRACT

The invention provides gold-plated mesoporous silicate bodies comprising pores and at least one agent and methods of using those bodies.

### CROSS-REFERENCE TO RELATED APPLICATIONS

This application claims the benefit of the filing date of U.S. application Ser. No. 61/567,477, filed on Dec. 6, 2011, the disclosure of which is incorporated by reference herein.

### BACKGROUND

The application of nanotechnology to biological sciences has brought a revolution in many areas because of the unique characteristics and potentials of nanoparticles (NPs). The main applications of nanobiotechnology include biological sensing, imaging, cell targeting and drug delivery, among others (O'Farrell et al., 2006; Stone et al., 2011; Salata, 2004; Slowing et al., 2010). Nanotechnology has been expanding into animal and human biology research mainly because cells can readily uptake NPs. However, the utility of NPs in plant science generally remains limited due to a characteristic architectural feature of the plant cells, the cell wall, which restricts their uptake (Rico et al., 2011). This could be the reason why the convergence between nanotechnology and plant biology has been limited to passive uptake, monitoring and phytotoxicity of different NPs in plants (Nair et al., 2010; Ma et al., 2010) or the biological synthesis of NPs (Thakkar et al., 2010).

Even though bombardment (Grichko et al., 2006; Torney et al., 2007), injection (Gonzalez-Melendi et al., 2008; Corredor et al., 2009) and ultrasonication (Wang et al., 2011; Liu et al., 2008) methods have been used for NP delivery into plant cells, the most common methods involve passive introduction, such as leaf uptake (Corredor et al., 2009; Birbaum et al., 2010; Eichert et al., 2008), protoplast or tissue incubation and root uptake (Liu et al., 2009; Pasupathy et al, 2008; Silva et al., 2010; U.S. Publication 2011/0059529; Serag et al., 2010; Yu et al., 2006; Lucas et al., 2010; Ravindran et al., 2005; Etxeberria et al., 2006; Onelli et al., 2008; Wild et al., 2009; Lin et al., 2009; Wang et al., 2009; Cifuentes et al., 2010; Khodakovskaya et al., 2011). Because the passive uptake processes of NPs can vary depending on the size (Gonzalez-Melendi et al., 2008; Corredor et al., 2009; Birbaum et al., 2010; Eichert et al., 2008; Perez-Donoso et al., 2010) and properties (Chen et al., 2010) of NPs and the types of plant tissues or cell structures (Zhu et al., 2008), it is challenging to control the delivery and function of the NPs in the particular tissues or cells that are targeted.

One of the most powerful tools for plant biotechnologists is the biolistic (biological ballistics) system for plant genetic transformation. This method has been used for the delivery of DNA into nuclear or plastid genomes of multiple plant species (Warzecha et al., 2010; Klein, 2011). A gene gun allows for the mechanical introduction of DNA-coated microcarriers, made of solid tungsten or gold with diameters ranging between 0.4-1.5 μm, into plant cells. The introduction of these microcarriers inside plant cells through bombardment relies on the acceleration during the shot and therefore, is dependent on their size and density. Bombardment can be considered an attractive alternative to passive NP uptake methods and, in fact, gene guns have been used to introduce NPs into animal (Clark et al., 1999; Lee et al., 2008; Svarovsky et al., 2009) and plant (Grichko et al., 2006; Torney et al., 2007) cell systems. For example, nanodiamonds were used with banana fruits (Grichko et al., 2006).

There are few methods for protein delivery to plant cells, none of them NP mediated, including microinjection (Staiger et al., 1994; Wymer et al., 2001) and cell-penetrating peptides (Chang et al., 2007; Chugh et al., 2008; Lu et al., 2010). While these methodologies have been used to introduce model proteins into plant cells, they require the skillful handling of cell materials and lack the protection needed for the introduced protein during the process. For instance, Wu et al. (2011) delivered a DNA-enzyme complex into plant cells using 1 μm gold microparticles through the biolistic method. The codelivery of the complex led to enhanced plant transformation efficiency but required covalent modification of the protein so that it would remain attached to the gold microparticle during bombardment.

### SUMMARY

As described hereinbelow, biolistic-mediated delivery of mesoporous silica nanoparticles (MSNs) and DNA to plant cells was performed via two strategies: gold plating the surfaces of MSNs (“gold functionalized MSNs”) to increase momentum during bombardment, e.g., by increasing overall density and without substantially altering the porous nature of the MSNs, and cobombardment of the MSNs with 0.6 μm gold particles. In both cases, a CaCl₂/spermidine-based protocol was used to coat DNA onto the particles. Biolistic delivery of MSN materials was improved by increasing the density of MSNs through gold plating. Furthermore, NP delivery was dramatically improved when the particles were combined with 0.6 μm gold particles during bombardment. Thus, the present invention provides systems for the efficient delivery of NPs into plant cells using biolistic methods. Additionally, the DNA-coating protocol enhanced the NP-mediated DNA delivery of MSNs and gold nanorods to plant cells.

In one embodiment, the invention provides a gold-plated mesoporous silicate body comprising pores and at least one agent, wherein the at least one agent is associated with the gold-plated mesoporous silicate body surface or embedded in the pores. In one embodiment, the gold-plated surface comprises a functional group. In one embodiment, the functional group comprises a primary, secondary, or tertiary amine, an amino acid or a peptide, an ionic liquid and derivatives thereof, or an amine terminating polymer. In one embodiment, the silicate body is a nanoparticle. In one embodiment, the silicate body is about 300 nm to about 900 nm in diameter.

The invention includes other gold-plated nanomaterials, including but not limited to gold-plated mesoporous carbon bodies, mesoporous polymer bodies, carbon nanotubes, or mesoporous metal oxide bodies, as well as other metal-plated nanomaterials, e.g., tungsten, palladium, platinum or iridium plated mesoporous silicate bodies, mesoporous carbon bodies, mesoporous polymer bodies, carbon nanotubes, or mesoporous metal oxide bodies.

In one embodiment, the metal-, e.g., gold-, plated surface comprises about 5 wt % or more of the metal-plated nanomaterial body, for instance, the metal-plated mesoporous silicate body. In one embodiment, the metal-plated surface comprises about 10 wt % or more, e.g., about 20 wt %, about 30 wt % or about 40 wt %, of the metal-plated nanomaterial body. In one embodiment, the invention provides metal-plated nanomaterials comprising pores and an agent that is associated with the nanomaterial surface but is not embedded in pores. In one embodiment, the invention provides a metal-plated nanomaterial body comprising pores and an agent that is embedded in the pores but is not associated with the nanomaterial surface. In one embodiment, the invention provides a metal-plated nanomaterial body comprising pores and two different agents, e.g., each of which is bioactive, where one of the agents is associated with the surface and the other is embedded in the pores. In one embodiment, a mixture of two or more different agents is embedded in the pores. In one embodiment, a mixture of two or more different agents is associated with the surface. In one embodiment, the invention provides a metal-plated nanomaterial body comprising pores and an agent that is associated with the surface and is embedded in the pores. As used herein, “embedded” in pores does not include physically restraining an agent in the pores, e.g., using a cap. In one embodiment, the body is a nanoparticle. In one embodiment, the body is about 50 nm to about 1600 nm, about 100 nm to about 900 nm, about 400 nm to about 800 nm, or about 500 nm to about 700 nm, in diameter. In one embodiment, the pores have a diameter of about 1 nm to about 100 nm, about 5 nm to about 50 nm, or about 7 nm to about 20 nm. In one embodiment, the body comprises two different agents, one of which is in the pores and the other of which is on the metal plate or attached to the surface.

In one embodiment, the metal-, e.g., gold-, plated surface comprises about 5 wt % or more of the metal-plated mesoporous silicate body (both on the surface and in the pores). In one embodiment, the gold-plated surface comprises about 10 wt % or more, e.g., about 20 wt %, about 30 wt % or about 40 wt %, of the gold-plated mesoporous silicate body (both on the surface and in the pores). In one embodiment, the invention provides a gold-plated mesoporous silicate body comprising pores and an agent that is associated with the mesoporous silicate body surface but is not embedded in the pores. In one embodiment, the agent comprises one or more of nucleic acid, a protein such as an enzyme, an antibacterial agent, an antifungal agent, an antiviral agent, or a hormone. In one embodiment, the protein is an antibacterial agent, an antifungal agent, an antiviral agent, or a hormone. In one embodiment, the invention provides a gold-plated mesoporous silicate body comprising pores and an agent that is embedded in the pores but is not associated with the mesoporous silicate body surface. In one embodiment, the invention provides a gold-plated mesoporous silicate body comprising pores and two different agents, e.g., each of which is bioactive, where one of the agents is associated with the mesoporous silicate body surface and the other is embedded in the pores. In one embodiment, a mixture of two or more different agents is embedded in the pores. In one embodiment, a mixture of two or more different agents is associated with mesoporous silicate body surface. In one embodiment, the invention provides a gold-plated mesoporous silicate body comprising pores and an agent that is associated with the mesoporous silicate surface and is embedded in the pores. As used herein, “embedded” in pores does not include physically restraining an agent in the pores, e.g., using a cap. In one embodiment, the silicate body is a nanoparticle. In one embodiment, the mesoporous silicate body is about 50 nm to about 1600 nm, about 100 nm to about 900 nm, about 400 nm to about 800 nm, or about 500 nm to about 700 nm, in diameter. In one embodiment, the pores have a diameter of about 1 nm to about 100 nm, about 5 nm to about 50 nm, or about 7 nm to about 20 nm. In one embodiment, the silicate body comprises two different agents, one of which is in the pores and the other of which is on the gold plate or attached to the mesoporous silicate body surface.

As disclosed hereinbelow, a gold nanoparticle functionalized mesoporous silica nanoparticle (Au-MSN) was employed for delivery of nucleic acid and codelivery of proteins, e.g., fluorescently labeled bovine serum albumin (BSA) and enhanced green fluorescent protein (eGFP), and plasmid DNA, to plant tissues using a biolistic particle delivery. Au-MSN with an average pore diameter of about 10 nm were shown to deliver and subsequently release DNA, and proteins and plasmid DNA, to the same cell after passing through the plant cell wall upon bombardment. Release of fluorescent eGFP indicates the delivery of active, non-denatured proteins to plant cells.

Both noncovalent and covalent associations of an agent with the mesoporous silicate surface are envisioned. In one embodiment, one of the agents comprises nucleic acid. In one embodiment, the nucleic acid encodes a protein. In one embodiment, the nucleic acid is microRNA, siRNA or other inhibitory RNA molecule. In one embodiment, one of the agents comprises protein. In one embodiment, one of the agents comprises an enzyme, an antibacterial agent, an antifungal agent, an antiviral agent, or a hormone, or any combinations thereof. In one embodiment, one of the agents comprises an enzyme, an antibacterial agent, an antifungal agent, or a hormone, a growth factor, an antigen, an antibody, a polypeptide, a peptide nucleic acid, and the like, or any combinations thereof. In one embodiment, one of the agents comprises an inorganic substance, an organic substance, an oligonucleotide (e.g., one having 50 or fewer nucleotides), a polynucleotide, a chimeric oligonucleotide, a polysaccharide, a lipid, an antibiotics, a ligand, a vitamin, a metabolite, an inducer and the like, or any combination thereof. In one embodiment, the pores comprise a mixture of agents, e.g., different proteins. In one embodiment, the surface of the gold-plated mesoporous silicate body comprises a mixture of agents, e.g., different nucleic acid molecules. In one embodiment, the agent(s) is precipitated onto the gold-plated surface. In one embodiment, the agent(s) is associated with the gold-plated mesoporous silicate body via electrostatic interactions, e.g., after functionalization. In one embodiment, the agent(s) is associated with the gold-plated mesoporous silicate body via covalent interactions, e.g., after functionalization. For example, with respect to proteins, there are four protein chemical targets that account for the vast majority of cross-linking techniques: 1) primary amines (—NH2), which exist at the N-terminus of each polypeptide chain (called the alpha-amine) and in the side chain of lysine residues (called the epsilon-amine); 2) carboxyls (—COOH), which exist at the C-terminus of each polypeptide chain and in the side chains of aspartic acid and glutamic acid; 3) sulfhydryls (—SH), which exist in the side chain of cysteine; and 4) carbonyls (—CHO) where ketone or aldehyde groups can be created in glycoproteins by oxidizing the polysaccharide post-translational modifications (glycosylation) with sodium meta-periodate. Any of those groups may be employed to link proteins to the surface of a particle, e.g., one that is functionalized, using coupling reactions such as a maleimide reaction.

In one embodiment, the covalent association is via a peptide having a protease cleavage site. In one embodiment, the covalent association is via a disulfide bond.

Also provided is a method of preparing a composition comprising gold-plated mesoporous silicate body comprising two different agents. The method includes providing a first composition comprising a plurality of gold-plated mesoporous silicate bodies comprising pores and a second composition comprising a solution, e.g., an ethanol solution, phosphate buffered saline, or a cell culture medium without growth factors, having a first agent. The first composition and second composition are mixed under conditions that allow for the first agent to enter the pores, thereby providing a third composition comprising gold-plated mesoporous silicate body comprising a first agent embedded in the pores. The third composition is contacted with a fourth composition comprising a solution having a second agent (which is different than the first agent) under conditions that allow for an association between the second agent and the surface of the mesoporous silicate body, thereby providing a composition comprising a gold-plated mesoporous silicate body comprising two different agents. In one embodiment, the third composition is dried prior to contact with the fourth composition. In one embodiment, the first agent is a complex of protein and nucleic acid and the second agent comprises nucleic acid that is the same as the nucleic acid in the complex. In one embodiment, the first agent is a complex of protein and nucleic acid and the second agent comprises nucleic acid that is different than the nucleic acid in the complex.

Also provided is a composition comprising a complex comprising a mesoporous silicate body comprising pores and a first agent, a calcium salt, e.g., calcium chloride, a carrier, e.g., a polyamine such as spermidine, gold particles, and a second agent. In one embodiment, the carrier comprises spermidine. In one embodiment, the gold particles have a diameter of about 0.4 μm to about 1.5 μm. In one embodiment, the gold particles have a diameter of about 0.2 μm to about 1.6 μm. In one embodiment, the gold particles have a diameter of about 0.4 μm to about 0.8 μm. In one embodiment, the first agent and the second agent are different. In one embodiment, one of the agents comprises nucleic acid.

Further provided is a method to deliver an agent to an eukaryotic cell, e.g., a mammalian cell, such as a human cell, an avian cell, a plant cell, an algal cell, or a fungal cell, or to a tissue, e.g., mucosal tissue, or an organ in a multi-cellular organism. The method comprises providing a composition having a gold-plated mesoporous silicate body and at least one agent, or a composition comprising gold particles and a mesoporous silicate body comprising pores and at least one agent, wherein the at least one agent is associated with the mesoporous silicate body surface or embedded in the pores of the silicate body. The composition is biolistically delivered to cells, a tissue or organ in an amount and under conditions effective to deliver the at least one agent to the cell, tissue or organ. The cell may be a plant cell, i.e., a dicot cell or a monocot cell. In one embodiment, the at least one agent comprises nucleic acid, a hormone, an antifungal agent, an antiviral agent, or a nutrient. In one embodiment, the agent is isolated DNA, e.g., on a plasmid.

The methods and compositions described herein are useful in genetic transformation, gene targeting, e.g., to assess loss or gain of function of different forms of a protein, such as different isoforms including different post-translationally modified forms of a protein, and protein interactions with other biomolecules. The direct delivery of a protein that directly or indirectly acts on a co-delivered nucleic acid molecule allows for direct genome modification that is simplified relative to techniques that use crosses between plants to introduce two genes/gene products to the same plant, that deliver a gene to a genetically modified plant or that deliver two genes to a plant.

### BRIEF DESCRIPTION OF THE FIGURES

FIG. 1. A) Schemes of 3 different classes of MSN and gold nanorods (NR). From left to right: MSN-10, and the PAMAM surface functionalized MSN (PAMAM-MSN-10); gold capped MSN-10 (Au^{Capped}-MSN-10) and FITC labeled MSN (FITC-Au^{Capped}-MSN-10); 1×, 2× or 3× gold-plated MSN-10 (1×, 2× or 3×Au^{Plated}-MSN-10) and ionic liquid surface functionalized MSN (IL-1×Au^{Plated}-MSN-10); gold nanorod (Au NR). B) SEM image of MSN-10. C) TEM image of Au^{Plated}-MSN-10. D) STEM image of Au^{Plated}-MSN-10.

FIG. 2. A) Agarose gel electrophoresis images of DNA-MSN complexation experiments of PAMAM-MSN-10, 1×Au^{Plated}-MSN-10 and IL-1×Au^{Plated}-MSN-10 at different ratios with 1 μg of DNA after 1 hour incubation at room temperature (RT). B) Agarose gel electrophoresis of the comparison of the CaCl₂/Spermidine (CaCl₂/Spe) and incubation DNA coating protocols done with 1 μg of plasmid DNA and 100 μg of 1×Au^{Plated}-MSN-10 or IL-1×Au^{Plated}-MSN-10. The pellet (DNA coated MSN) or the supernatant (S/N, free DNA) were loaded for each procedure.

FIG. 3. A) Differences in transient marker gene expression between the DNA-MSN incubation (Incubation) and CaCl₂/Spe DNA coating protocol (Coating) procedures onto 1×Au^{Plated}-MSN-10 (Plain) and IL-1×Au^{Plated}-MSN-10 (Ionic Liquid). Number of red fluorescent cells per sample (2 cm×3.5 cm onion epidermal tissue) was scored one day after bombardment using a 10× objective of Zeiss Axiostar plus microscope. B) Bright field and fluorescence images taken with a 10× objective of Zeiss Axiostar plus microscope of plant tissues 1 day after bombardment with different types of NPs coated with GFP or mCherry expressing plasmid DNA using the CaCl₂/Spe coating protocol. From left to right: mCherry expressing onion epidermis cell after bombardment with gold nanorods; GFP expressing maize leaf cell after bombardment with Au^{Capped}-MSN-10; Tobacco leaf cells expressing GFP after bombardment with FITC-Au^{Capped}-MSN-10. Bar=100 μm.

FIG. 4. A) Effects of rupture disk types on MSN-10 and 3×Au^{Plated}-MSN-10 delivery efficiency. B) Effects of gold plating rounds (MSN-10, 1×, 2× and 3×Au^{Plated}-MSN-10) on delivery efficiency. C) Comparison of the bombardment performance of Au^{Capped}-MSN-10 and 1×Au^{Plated}-MSN-10.mCherry expressing plasmid was used in all experiments. Number of red fluorescent cells per sample (2 cm×3.5 cm onion epidermal tissue) was scored one day after bombardment using a 10× objective of Zeiss Axiostar plus microscope. Bars in the graphs labeled with different letters indicate significantly different means according to Duncan's New Multiple Range Test (α=0.05).

FIG. 5. A) Schemes showing NP (Au^{Capped}-MSN-10 or gold NRs)-GP co-bombardment treatments (left) and graph showing the effects in NPs delivery to onion epidermis cells (right). Treatment #1 (S1:cNP/S2:cNP): two shots with mCherry expressing plasmid coated NPs. Treatment #2 (S1:GP/S2:cNP): first shot with uncoated 0.6 μm gold and second shot with coated NPs. Treatment #3 (S1:GP+cNP): the macrocarrier was loaded first with an aliquot of uncoated 0.6 μm gold and after the DNA coated NPs. One shot of this mixture was bombarded to plant tissues. Treatment #4 (S1:(c(cNP)GP)): one shot with a double DNA coating procedure, first mCherry expressing plasmid is coated onto NPs and then a second coating procedure is made to the mixture of these particles, 0.6 μm gold and GFP expressing plasmid. S1: shot 1; S2: shot 2; NP: nanoparticle; GP: 0.6 μm gold; cNP, cNR and cMSN: DNA coated NP, NR or MSN respectively. In Treatments #2 and 3 where uncoated 0.6 μm gold was used, a 2 μL aliquot of 0.6 μm gold (from a 30 μg μL⁻¹ in sterile ddH₂O stock) was centrifuged at 5000 rpm, the supernatant removed and the pellet resuspended in 5 μL of ethanol and loaded in the macrocarrier. B) Bright field and fluorescence images of onion epidermis cells expressing GFP and mCherry after co-bombardment with the Au^{Cappd}-MSN-10 and 0.6 μm gold complex c(c(Au^{Capped}-MSN-10)GP). Bar=100 μm. C) TEM image of the (c(cAu^{Capped}-MSN-10)GP) complex. D) Onion epidermis tissue bombarded with S1:c(FITC-Au^{Capped}-MSN-10+GP) complex. Bright field image on the left and then subsequent fluorescence images of a Z stack of the tissue in which focused MSN can be seen along the depth of the cell. Bar=10 μm, distance between Z stack images=1.5 μm. E) Two layers in different depth levels of a mCherry expressing onion epidermis cell after bombardment with S1:c(NR+GP) complex. For each layer the fluorescence image (left) and an amplified image corresponding to the white square of the 2 consecutive and rotated (0 and 90°) DIC images are shown. In these images several nanorods (pointed with white arrows) can be detected by the change on light emission on the rotated images. Bar in fluorescence images=50 μm; in DIC images=1 μm.

FIG. 6. Schematic representation of Au-MSN mediated co-delivery of proteins and plasmid DNA to plant cells via particle bombardment.

FIG. 7. BET nitrogen sorption isotherms (A) and BJH pore size distribution (B) of MSN (red) and Au-MSN (black).

FIG. 8. TEM image (A), STEM image (B) and SEM image (C) of Au-MSN. X-ray diffraction patterns of MSN (red) and Au-MSN (black) (D). TEM and STEM images were obtained using a Tecnai F² microscope and the SEM was obtained using a Hitachi S4700 FE-SEM system with a 10 kV accelerating voltage.

FIG. 9. Transmission electron micrographs for direct comparison of MSN (A) and Au-MSN (B).

FIG. 10. Normalized release profiles of FITC-BSA (green), TRITC-BSA (red), and eGFP (blue) from Au-MSN in pH 7.4 PBS solution.

FIG. 11. Scanning transmission electron microscopy image (A) and energy dispersive X-ray (EDX) spectrum (B) of Au-MSN. Red box in (A) is the area that was scanned for EDX analysis. The source of the copper detected by the EDX is the TEM grid.

FIG. 12. Delivery of proteins into plant tissues. Bright field and green channel images of A) onion epidermis cells showing FITC-BSA release 30 minutes after bombardment. B) Intracellular release of eGFP in onion epidermis cells one day after bombardment. Tobacco (C) or teosinte (D) leaf cell showing FITC-BSA release one day after bombardment.

FIG. 13. High angle X-ray diffraction pattern for MSN (red) and Au-MSN (black).

FIG. 14. Association of Au-MSN and protein release in plant cells. Bright field, red channel, green channel, and merged images of an onion cell 1 day after bombardment with TRITC-BSA loaded, FITC labeled Au-MSN. White arrows point at Au-MSN clusters.

FIG. 15. Scanning electron micrographs of MSN-10 synthesized in different batches, demonstrating the consistency of the morphology and particle size between preparations.

FIG. 16. Bright field, green channel, and red channel fluorescent microscopy images of onion epidermis cells bombarded with empty Au-MSN (A), TRITC-BSA protein loaded and GFP expressing plasmid DNA coated Au-MSN (B), FITC-labeled BSA protein loaded and mCherry expressing plasmid DNA coated loaded Au-MSN (C), and eGFP protein loaded and mCherry expressing plasmid DNA coated Au-MSN (D).

### DETAILED DESCRIPTION

To date NP-mediated delivery of biogenic molecules to plant cells has been limited to nucleic acids, including double or single stranded DNA (Torney et al., 2007; Liu et al., 2008; Liu et al., 2009; Martin-Ortigosa et al., 2012; Pasupathy et al., 2008; Wang et al., 2011) and small interfering RNA (Silva et al., 2010). Delivery and release of chemical substances such as phenanthrene and plant growth regulators have also been reported (Grichko et al., 2006; Wild et al., 2009). Using the interior pore volume and the exterior surface of MSN along with particle bombardment technology, plasmid DNA carrying a chemically inducible marker gene encoding for green fluorescent protein (GFP) and a chemical inducer (β-oestradiol) was co-delivered to plant tissues (Torney et al., 2007). The controlled release of β-oestradiol led to the expression of GFP in plant cells (Torney et al., 2007). Additionally, MSN delivery to plants through the biolistic method was improved by increasing the density of MSN by gold functionalization; leading to an enhanced cell penetration and subsequent DNA expression (Martin-Ortigosa et al., 2012).

### DEFINITIONS

The term “amino acid,” comprises the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyro sine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarco sine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide). Other suitable amino and carboxy protecting groups are known to those skilled in the art (See for example, T. W. Greene, Protecting Groups In Organic Synthesis; Wiley: New York, 1981, and references cited therein).

The term “polypeptide” describes a sequence of at least 50 amino acids (e.g., as defined hereinabove) or peptidyl residues while a peptide describes a sequence of at least 2 and up to 50 amino acid residues. The sequence may be linear or cyclic. For example, a cyclic peptide can be prepared or may result from the formation of disulfide bridges between two cysteine residues in a sequence. A polypeptide can be linked to other molecules through the carboxy terminus, the amino terminus, or through any other convenient point of attachment, such as, for example, through the sulfur of a cysteine. In one embodiment of the invention a polypeptide comprises about 50 to about 300 amino acids. In another embodiment a peptide has about 5 to about 25 amino acids Peptide and polypeptide derivatives can be prepared as disclosed in U.S. Pat. Nos. 4,612,302; 4,853,371; and 4,684,620, or as described in the Examples hereinbelow. Polypeptide sequences specifically recited herein are written with the amino terminus on the left and the carboxy terminus on the right.

The term “nucleic acid”, “polynucleic acid” or “polynucleic acid segment” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991; Ohtsuka et al., 1985; Rossolini et al., 1994). An “oligonucleotide” typically includes 30 or fewer nucleotides.

As used herein, the terms “isolated and/or purified” refer to in vitro preparation, isolation and/or purification of a nucleic acid or protein (polypeptide or peptide) so that it is not associated with in vivo substances, or is substantially purified from in vitro substances. Thus, with respect to an “isolated nucleic acid molecule”, which includes a polynucleotide of genomic, cDNA, or synthetic origin or some combination thereof, or an “isolated polypeptide or peptide”, the “isolated nucleic acid molecule” or “isolated polypeptide or peptide” (1) is not associated with all or a portion of cell based molecules with which the “isolated nucleic acid molecule” or “isolated polypeptide or peptide” is found in nature, (2) is operably linked to a molecule which it is not linked to in nature, or (3) does not occur in nature as part of a larger sequence. An isolated nucleic acid molecule means a polymeric form of nucleotides of at least 10 bases in length, either ribonucleotides or deoxynucleotides or a modified form of either type of nucleotide. The term includes single and double stranded forms of DNA. The term “oligonucleotide” referred to herein includes naturally occurring, and modified nucleotides linked together by naturally occurring, and non-naturally occurring oligonucleotide linkages. Oligonucleotides are a polynucleotide subset with 200 bases or fewer in length. In one embodiment, oligonucleotides are 10 to 60 bases in length including 12, 13, 14, 15, 16, 17, 18, 19, or 20 to 40 bases in length. Oligonucleotides may be usually single or double stranded. Oligonucleotides can be either sense or antisense oligonucleotides. The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like.

The term “complexed” refers to binding of a molecule to a mesoporous silicate body, typically through means other than covalent bonding. Such binding can take the form of, e.g., ionic or electrostatic interactions, or other attractive forces.

Exemplary Agents and Compositions for Delivery to Plant Cells.

Many molecules are unable to cross the membrane barrier of cells without the assistance of transport systems. For example, proteins are generally unable to cross the membrane barrier of cells without the assistance of protein transport systems (Jung et al., 2009). This challenge has led to the development of protein delivery systems using nanoparticle materials including polymers (Lee et al., 2010), carbon nanotubes (Kam et al., 2005) and mesoporous silica nanoparticles (MSN) (Slowing et al., 2007; Bale et al., 2010). The MSN materials have many beneficial characteristics for protein delivery, including large pore volume, mechanically and chemically stable framework, tunable pore sizes, and chemically functionalizable surfaces that make them ideal to host guest molecules of various sizes and shapes (Slowing et al., 2008; Slowing et al., 2010). Additionally, MSN materials offer distinct advantages over other nanoparticle systems by protecting proteins from denaturation and maintaining protein activity in various environments when encapsulated in the porous framework (Slowing et al., 2007; Trewyn et al., 2007). Recently, MSN material has been successfully used for protein encapsulation and in vivo release in mammalian cell systems (Slowing et al., 2007; Bale et al., 2010).

There are a few examples of protein delivery methodologies to plant cells, such as microinjection (Staiger et al., 1994; Wymer et al., 2001) and cell penetrating peptides (Chang et al., 2007; Chugh et al., 2008; Lu et al., 2010; Qi et al., 2011). While these methodologies could be used to introduce model proteins into plant cells, they have major disadvantages including the requirement of skillful handling of cell materials or lack of protection of the introduced protein during the process. Recently, using particle bombardment, Wu et al. (2011) delivered a DNA-enzyme complex (a transposase covalently linked to 1-μm gold particles and subsequently coupled with transposon containing DNA fragments) into plant cells. The co-delivery of DNA and enzyme in this case led to enhanced plant transformation efficiency.

Delivery of bioactive, e.g., proteins, or co-delivery of bioactive agents, such as proteins and DNA, to plant cells has great biological significance. Thus, with respect to delivery of protein and nucleic acid, in addition to the potential of enhancing genetic transformation and gene targeting in plants (Wu et al., 2011), researchers could assess loss or gain of function of different post-translationally modified forms of a protein, and protein interactions with other biomolecules. Also, direct delivery and release of proteins in plant cells could facilitate the understanding of cellular machinery or signal pathways more effectively. For example, this would allow for a greater understanding of protein functions in host cells where protein production pathways are impaired, or analyzing cellular regulatory functions through delivery of antibodies (Trewyn et al., 2007; Wu et al., 2011; Lim et al., 2009; Shah et al., 2011).

Nanoparticle mediated delivery of bioactive (biogenic) molecules to plant cells, such as double or single stranded DNA (Pasupathy et al., 2008; Liu et al., 2008; Wang et al., 2011; Liu et al., 2009; Torney et al., 2007) and small interfering RNA (Silva et al., 2010), and delivery and release of chemical substances such as phenanthrene and plant growth regulators (Wild et al., 2009; Grichko et al., 2006), have been reported. However, biolistic methods to deliver agents in an effective amount to intact plant cells depend on the density of the delivery vehicle and the loading capacity of the vehicle. The present invention provides for mesoporous silicate particles with sufficient density for biolistic delivery and enhanced loading capacity.

The gold-plated MSNs of the invention are generally useful for delivering one or more agents to plant cells. The nature of the agents is not critical. The agents include one or more of genes, nutrients (vitamins, etc.), and/or biocidal or pesticidal agents (e.g., insecticides or herbicides). For example, the term includes but is not limited to antibacterial agents, antifungal agents, antiviral agents, polypeptides, hormones, enzymes, antibodies, and RNA or DNA molecules of any suitable length, or any combination thereof. For instance, the RNA or DNA molecules may encode herbicide resistance, drought tolerance, a polypeptide associated with enhanced nutritional value, and the like.

Exemplary agents for delivery to cells, including plant cells, include but are not limited to one or more of polypeptides, and/or polynucleotides (DNA or RNA) encoding a screenable marker, a polypeptide that can enhance or stimulate cell growth, an enzyme such as a recombinase, an integrase, a site-specific recombinase, a DNA topoisomerase, an endonuclease, a zinc-finger nuclease, a Transcription Activator-Like (TAL) effector nuclease, a homing endonuclease, a transposase, a meganuclease, a restriction enzyme, a DNA polymerase, a DNA ligase, and the like, a transcription factor, a repressor, a DNA binding protein, including but not limited to a zinc-finger protein, a TAL Effector protein, a DNA repair protein, a transactivating factor, leucine-zipper protein, a cell cycle protein, a RNA binding protein, including but not limited to a DICER, a DICER-LIKE protein, a Drosha, a Rnase, a RNA-dependent RNA polymerase, ribosomal proteins, and the like.

In some examples the agent comprises a polynucleotide or polypeptide that stimulates cell growth. The agent employed in compositions for delivery to cells may provide a means for positive selection of recipient target cells, increased transformation efficiency, increased plastid transformation efficiency, increased gene targeting or combinations thereof. Genes that enhance or stimulate cell growth include genes involved in transcriptional regulation, homeotic gene regulation, stem cell maintenance and proliferation, cell cycle regulation, cell division, and/or cell differentiation such as WOX family genes including WUS homologues (Mayer et al., 1998; WO01/0023575; U.S. Publication 2004/0166563); aintegumenta (ANT) (Klucher et al., 1996; Elliott et al., 1996; GenBank Accession Nos. U40256, U41339, Z47554); clavata (e.g., CLV1, CVL2, CLV3) (WO03/093450; Clark et al., 1997; Jeong et al., 1999; Fletcher et al., 1999); Clavata and Embryo Surround region genes (e.g., CLE) (Sharma et al., 2003; Hobe et al., 2003; Cock & McCormick, 2001; Casamitjana-Martinez et al., 2003); babyboom (e.g., BNM3, BBM, ODP1, ODP2) (WO00/75530; Boutileir et al., 2002; Zwille (Lynn et al., 1999); leafy cotyledon (e.g., Lec1, Lec2) (Lotan et al., 1998; WO00/28058; Stone et al., 2001; U.S. Pat. No. 6,492,577); Shoot Meristem-less (STM) (Long et al., 1996); ultrapetala (ULT) (Fletcher, 2001); mitogen activated protein kinase (MAPK) (Jonak et al., 2002); kinase associated protein phosphatase (KAPP) (Williams et al., 1997; Trotochaud et al., 1999); ROP GTPase (Wu et al., 2001; Trotochaud et al., 1999); fasciata (e.g. FAS1, FAS2) (Kaya et al., 2001); cell cycle genes (U.S. Pat. No. 6,518,487; WO99/61619; WO02/074909), Shepherd (SHD) (Ishiguro et al., 2002; Poltergeist (Yu et al., 2000; Yu et al., 2003); Pickle (PKL) (Ogas et al., 1999); knox genes (e.g., KN1, KNAT1) (Jackson et al., 1994; Lincoln et al., 1994; Venglat et al., 2002); fertilization independent endosperm (FIE) (Ohad et al., 1999), cell cycle regulators (e.g., cyclins, cyclin-dependent kinases (CDKs), and the like. In some examples, the polypeptide(s) that enhances or stimulates cell growth is a cell cycle regulator (e.g., cyclin, cyclin-dependent kinase), a wuschel polypeptide, a babyboom polypeptide, a knotted polypeptide, or any combination thereof.

The agent(s) can be free in the mesopores of the nanomaterial, e.g., silicate, body or can be associated (e.g., via covalent or noncovalent interactions) with the interior surface of the pores or the exterior of the body. When the agent(s) is/are free in the pores, it/they can typically be loaded by contacting a nanomaterial body, e.g., a mesoporous silicate body, in a solution of the agent. When the agent(s) is/are associated with the interior surface of the pores or the exterior of the body, it/they may be loaded by allowing the agent to react with, or be attracted to, groups on the interior surface of the pores, the exterior of the body, groups that functionalize the metal-, e.g., gold-, plated surface of the body, or under conditions suitable to allow the agent to associate with the body with or without the metal-plated surface, or any combination thereof. In one embodiment of the invention, the nanomaterial bodies, e.g., mesoporous silicate bodies, can be stirred in ethanol or other loading buffer, e.g., phosphate buffered saline, for a period of time sufficient to load the material into the pores. Any suitable and effective solvent can be employed in this particular manner of pore loading.

In one embodiment of the invention, an agent can be “associated” with the metal-plated surface or functionalized metal-plated surface of mesoporous silicates through ionic, covalent or other bonds (e.g., electrostatic interactions). For example, DNA molecules can be associated with mesoporous silicates of the invention through ionic, covalent or other bonds (e.g., electrostatic interactions). The polyanionic nature of plasmid DNAs or genes makes them electrostatically attracted to positively charged molecules, although certain conditions allow for nucleic acid to associate with surfaces that are not necessarily positively charged.

### Mesoporous Silicates

Mesoporous silicates typically may have a particle size of about 50 nm to about 1 μm. In one embodiment, the mesoporous silicates may have a particle size of at least about 100 nm. In another embodiment, the mesoporous silicates may have a particle size of less than about 1600 nm, e.g., a diameter of less than about 1600 nm. In one embodiment, the mesoporous silicates may have a particle size of about 250 nm. In other embodiments, the mesoporous silicate body may be a sphere having a diameter of about 50 to about 150 nm, about 60 to about 300 nm, or about 250 to about 1000 nm. The mesoporous silicates may be spherical or may have other shapes, such as rods. In certain embodiments, the mesoporous silicate body can be a rod having a length of about 50 to about 150 nm, about 60 to about 300 nm, or about 250 to about 1500 nm. The mesoporous silicates for use in the metal-plated nanomaterials, e.g., MSNs, may be of any shape and size, provided the pore structure is suitable for receiving and entrapping an agent.

The pores typically have a diameter of from about 1 to about 100 nm. In one embodiment of the invention, the pores have a diameter of at least about 2 nm. In another embodiment, the pores have a diameter of about 1 nm to about 20 nm, or about 5 nm to about 15 nm. In other embodiments, the pores have diameters of greater than about 10 nm, or greater than about 15 nm. Typically, the pores have a diameter of less than about 75 nm or less than about 25 nm.

Mesoporous silicate particles may be prepared by various methods such as by co-condensing one or more tetraalkoxy-silanes and one or more organo-substituted trialkoxy-silanes to provide a population of mesoporous silicate particles having monodisperse particle sizes and preselected particle shapes, wherein the substituted trialkoxy-silane is not a co-solvent. The mesoporous silicate particles can be prepared by co-condensing one or more tetraalkoxy-silanes and one or more (3-cyanopropyl)trialkoxy-silanes to provide the mesoporous silicate particles as nanorods. Any suitable and effective tetraalkoxy-silane and alkyl-trialkoxy-silane can be employed. Many such silanes are described in, e.g., Aldrich Handbook of Fine Chemicals, 2003-2004 (Milwaukee, Wis.).

The mesoporous silicates may be prepared from surfactant micelles of C₁₀-C₁₆ alkyl(trialkyl)ammonium salts in water, followed by introduction into the solution of an alkyl ortho silicate, such as tetraethylortho silicate (TEOS), and one or more functionalized silanes, such as one or more mercaptoalkyl-, chloroalkyl-, isocyanate-, aminoalkyl-, carboxyalkyl-, sulfonylalkyl-, arylalkyl-, alkynyl-, or alkenyl-silanes, wherein the (C₂-C₁₀)alkyl chain is optionally interrupted by —S—S—, amido (—C(═O)NR—), —O—, ester (—C(═O)O—), and the like. For example, functionalized silanes can be, e.g., 3-mercaptopropyl-trimethoxysilane (MPTMS) or 3-isocyanatoprypyl-triethoxysilane (ICPTES). The aqueous mixture is stirred at moderate temperatures until the silicate precipitates, and it is collected and dried. The surfactant “template” is then removed from the pores of the ordered silicate matrix, for example, by refluxing the silicate in aqueous-alcoholic HCl. The remaining solvent can be removed from the pores of the silicate by placing it under high vacuum. The polarity of the interior of the pores can also be adjusted by adding other functionalized silanes to the reaction mixture, including ones comprising non-polar inert groups such as aryl, perfluoroalkyl, alkyl, arylakyl and the like. The exterior of the silicate matrix can be functionalized by grafting organic moieties comprising functional groups thereto. These groups can in turn be employed to link the particles to other moieties.

Recent advancements in the synthesis of monodispersed, large average pore diameter mesoporous silica nanoparticle (MSN) materials with highly functionalizable surface area (≧400 m²g⁻¹) and pore volume (1.05 cm³g⁻¹) has led to the development of a series of biomolecule delivery vehicles, where various proteins, small DNA and RNA sequences, and other biomolecules are loaded into the mesopores and on the external surface, and released in vitro or in cellular systems (Kim et al., 2011; Li et al., 2011; Torney et al., 2007; Xia et al., 2009). The large pore volumes and surface area of these materials allow for the efficient adsorption of biomolecules and subsequent delivery to viable animal and plant cells. Additionally, recent reports on functionalizing the surface of MSN demonstrate that this material can be tuned to optimize various applications. Organic and inorganic functionalization leads to control in MSN uptake by cells (Slowing et al., 2006), magnetization of MSN (Giri et al., 2005), the DNA/RNA affinity for MSN (Solberg et al., 2006), and increasing the inherent density of MSN (Torney et al., 2007).

Delivery of biomolecules mediated by MSN materials is particularly interesting because proteins are often unable to cross the membrane barrier of cells without the assistance of protein transport systems (Jung et al., 2009). Several proteins have been successfully loaded and released from MSN materials (Bhattacharyya et al., 2010; Ho et al., 2008; Kim et al., 2010; Song et al., 2007; and Vivero-Escoto et al., 2010), however; only one example demonstrated the in vivo release of active protein from MSN in a mammalian cell system and no protein delivery to plant cells has been reported (Slowing et al., 2007).

The gold-plated MSNs of the invention are generally useful for delivering one or more agents to plant cells. The nature of the agents is not critical. The agents include genes, nutrients (vitamins, etc.), and biocidal or pesticidal agents (e.g., insecticides or herbicides). For example, the term includes but is not limited to antibacterial agents, antifungal agents, antiviral agents, polypeptides, hormones, enzymes, antibodies, and RNA or DNA molecules of any suitable length, or any combination thereof. For instance, the RNA or DNA molecules may encode herbicide resistance, drought tolerance, a polypeptide associated with enhanced nutritional value, and the like.

The agent(s) can be free in the mesopores of the silicate body or can be associated (e.g., covalently or noncovelantly bonded) with the interior surface of the pores or the exterior surface of the mesoporous silicate body. When the agent(s) is/are free in the pores, it/they can typically be loaded by contacting a mesoporous silicate in a solution of the agent. When the agent(s) is/are associated with the interior surface of the pores or the exterior surface of the gold-plated mesoporous silicate body, it/they may be loaded by allowing the agent to react with, or be attracted to, groups on the interior surface of the pores, the exterior surface of the mesoporous silicate body, or groups that functionalize the gold-plated surface under conditions suitable to allow the agent to associate. In one embodiment of the invention, the mesoporous silicates can be stirred in ethanol for a period of time sufficient to load the material into the pores. Any suitable and effective solvent can be employed in this particular manner of pore loading.

In one embodiment of the invention, an agent can be “associated” with the gold-plated surface or functionalized gold-plated surface of mesoporous silicates through ionic, covalent or other bonds (e.g., electrostatic interactions). For example, DNA molecules can be associated with mesoporous silicates of the invention through ionic, covalent or other bonds (e.g., electrostatic interactions). The polyanionic nature of plasmid DNAs or genes makes them electrostatically attracted to positively charged molecules, although certain conditions allow for nucleic acid to associate with surfaces that are not necessarily positively charged.

The invention will be further described by the following non-limiting examples.

### Example 1

The type of particle used in the biolistic method is one of the most important parameters that affects delivery (Zhang et al., 2007); therefore, one of the major challenges for the delivery of NPs to plant cells is their small size and low weight compared to any typical microcarriers used in plant transformation. For DNA delivery, small size and surface characteristics of NPs can also attribute to the inefficient delivery, due partially to poor binding/attachment of DNA to NPs. Most current protocols use simple NP and DNA incubation steps and, depending on the nature of the NPs, usually a surface functionalization step is required to promote binding (Slowing et al., 2010; Svarovsky et al., 2009). To overcome these problems, NP density, NP-DNA coating protocols, as well as parameters in the biolistic delivery system, were modified, such as: (1) increasing the density by gold plating MSN surfaces; (2) using a CaCl₂/spermidine DNA coating protocol for enhanced DNA-NPs attachment; (3) co-bombarding NPs with 0.6 μm gold microparticles (GPs) and DNA. These were tested over two distinct types of NPs used for different biological applications, MSN and gold nanorods (NRs).

### Experimental Section

Mesoporous Silica Nanoparticle (MSN) Synthesis:

All the MSN-10 used were synthesized as described previously (Kim et al., 2010). Briefly, the non-ionic surfactant Pluronic® P104 (7.0 g) was added to 1.6 M HCl (273.0 g). After stirring for 1 hour at 55° C., tetramethylorthosilicate (TMOS, 10.64 g) was added and stirred for an additional 24 hours. The resulting mixture was further hydrothermally treated for 24 hours at 150° C. in a high-pressure reactor. Upon cooling to room temperature (RT), the white solid was collected by filtration, washed with copious amounts of methanol and dried in air. To remove the surfactant P104, the silica material was heated to 550° C. at a ramp rate of 1.5° C. min⁻¹ and maintained at 550° C. for 6 hours. The fluorescein isothiocyanate (FITC) labeling of Au^{Capped}-MSN-10 was done by adding 5 mg (12.8 μmol) of FITC to 3-aminopropyltrimethoxysilane (APTMS, 13 μmol) in dry DMSO (0.5 mL) and stirred for 30 minutes, and then added to a toluene suspension (100 mL) of MSN-10 (1.0 g). The suspension was refluxed for 20 hours under nitrogen and the resulting material was filtered, washed with toluene and methanol, and dried under vacuum overnight.

For Au^{Capped}-MSN-10, 3-mercaptopropyltrimethoxysilane (MPTMS, 2 mmol) was grafted to MSN-10 (1.0 g) by refluxing in toluene (100 mL) for 20 hours under nitrogen. The resulting thiol-functional MSN (thiol-MSN-10) was filtered, washed with toluene and methanol, and dried under vacuum overnight. To activate thiol-MSN-10, 2,2-dipyridyldisulfide (3 mmol) was added to a methanol suspension of the thiol-MSN-10 and stirred for 24 hours in the absence of light. The resulting material was filtered, washed with methanol, and dried under vacuum overnight. To synthesize amine-linker-MSN-10 (2-(propyldisulfanyl)ethylamine-MSN-10), 2-aminoethanethiol (3 mmol) was added to a methanol suspension of the activated thiol-MSN-10 and stirred for 24 hours. The resulting material was filtered and washed with methanol, and dried under vacuum overnight. Carboxylic acid-functionalized gold NPs (25 mg, AuNP—COOH) were suspended in PBS solution (3 mL) along with N-Ethyl-N′-(3-dimethylaminopropyl)carbodiimide (EDC, 100 mg) and N-hydroxysuccinimide (NHS, 80 mg) and stirred for 15 minutes. Amine linker-MSN-10 (50 mg) was added to the mixture and stirred for 24 hours. The Au^{Capped}-MSN were collected by centrifugation, washed three times with water and then, resuspended in water (5 mL), frozen in liquid nitrogen and dehydrated in a lyophilizer.

For PAMAM-MSN-10 synthesis, 3-mercaptopropyltrimethoxysilane (MPTMS, 2 mmol) was added to a toluene suspension (100 mL) of MSN-10 (1.0 g) and refluxed for 20 hours under nitrogen, then filtered, washed with toluene and methanol, and dried under vacuum overnight. The thiol-MSN-10 was activated by the same method as previously described for the amine-linker-MSN-10, instead of 2-aminoethanethiol, an equimolar amount of 11-mercaptoundecanoic acid was added to a methanol suspension (acid-linker-MSN-10). Acid-linker-MSN-10 (20 mg) were suspended in PBS solution (3 mL) and EDC (100 mg), and N-hydroxysuccinimide (NHS, 80 mg) was added and stirred for 15 minutes. PAMAM-G4 dendrimer (25 mg) was added to the mixture, stirred for 24 hours. The particles were collected by centrifugation, washed three times with water, resuspended in water (5 mL), frozen in liquid nitrogen and dehydrated in a lyophilizer.

For the gold 1×Au^{Plated}-MSN-10, ethylenediamine (0.45 mL) was added to an aqueous solution of HAuCl₄.3H₂O (1.0 g) in water (10 mL), stirred for 30 minutes and followed by the addition of ethanol (70 mL). The resulting Au(en)₂Cl₃ precipitate was filtered, washed with ethanol, dried under vacuum and after that, 0.372 g was dissolved in H₂O (150 mL) and the pH adjusted to 10.0 using NaOH. MSN-10 (2 g) was added to the solution, the pH was readjusted to 9.0 with NaOH and stirred for 2 hours. The product was filtered and dried under vacuum for 2 days and then, reduced under H₂ flow at 150° C. for 3 hours. For the ionic liquid layer functionalization of IL-1×Au^{Plated}-MSN-10, 1-propyltriethoxysilane-3-methylimidazolium chloride (PMIm, 2 mmol) was added to a N,N-dimethylformamide suspension (DMF, 100 mL) of Au-MSN-10 (1.0 g) and then refluxed for 20 hours under nitrogen. The resulting material was filtered, washed with DMF and methanol, and dried under vacuum overnight. To synthesize 2× and 3×Au^{Plated}-MSN-10, 1×Au^{Plated}-MSN-10 was subjected to the Au(en)₂ impregnation and reduction process an additional one and two more cycles, respectively.

Zeta Potential Measurements:

Each sample (5 mg) was sonicated in PBS (10 mL) for 30 minutes. The samples were then analyzed on a Malvern Instruments Zetasizer.

MSN Surface Area and Porosity Measurement:

The surface area and average pore diameter were measured using N₂ adsorption/desorption measurements in a Micromeritics ASAP 2020 BET surface analyzer system. The data were evaluated using BET and BJH methods to calculate surface area and pore distributions, respectively. Samples were prepared by degassing at 100° C. overnight before analysis.

Nanorods:

gold nanorods (25 nm×73 nm, catalog number 30-25-700-100) were purchased from Nanopartz.

Plant Material:

Onion epidermis tissue was obtained from white bulk onion bulbs. The tissue was cut in 2 cm×3.5 cm rectangles and placed with the peeled face upwards in agar media (0.5 mM 2-(N-morpholino)ethanesulfonic acid (MES) pH 5.7, and 15 g L⁻¹ of BD Bacto™ agar, pH 5.7) or MS media (Murashige et al., 1962) (MS salts & vitamins from PhytoTechnology Laboratories, 2% sucrose, 2.5 mg L⁻¹ Phytagel™ from Sigma Aldrich, pH 5.7). Leaf pieces of 3 to 4 week old maize plants of the inbred A188 germinated in soil were cut in approximately 3 cm long pieces and placed with the adaxial surface up on MS media. Leaves from 6 to 8 week old in vitro-grown tobacco plants (Nicotiana tabacum var. Petite Havana) were placed with the adaxial surface up on MS media.

DNA-MSN Complexation Experiments:

MSN stocks at 10 mg mL⁻¹ in sterile double distilled water (ddH₂O) were sonicated in a water bath sonicator (FS6 from Fisher Scientific) for 15 seconds. One, 10, 30 or 50 μg of MSN were incubated with plasmid DNA (1 μg) in a final volume of 15 μL for 1 hour at RT. The total mixture was loaded in a 1% agarose gel stained with ethidium bromide and electrophoresed at 100 V for 25 minutes. The supernatant of the CaCl₂/Spe DNA coating protocol was subjected before loading to dialysis for 30 minutes using a MF™-Membrane filter of 0.025 μm (Millipore).

Nanoparticle DNA Coating and Sample Bombardment:

The plasmids ER-rk (Nelson et al., 2007) and pLMNC95 (Mankin et al., 2001) for mCherry and GFP expression, respectively, were obtained from the Arabidopsis Biological Resource Center (ABRC stocks CD3-959 and CD3-420, respectively, http://www.arabidopsis.org). The DNA coating and bombardment procedures onto NPs were done according to standard protocols (Klein et al., 1987; Sanford et al., 1993) with the following modifications (protocols described for one shot). One hundred μg of MSN (from a 10 μg μL⁻¹ stock in sterile ddH₂O) or 150 μL of the commercial nanorod suspension (previously washed with sterile ddH₂O after centrifuging at 2000 g for 6 minutes and resuspended in 10 μL of sterile ddH₂O) were sonicated for 15-30 seconds in a water bath sonicator. One μg of plasmid DNA (from a 250 ng μL⁻¹ stock in sterile ddH₂O), 12.5 μL of a 2.5 M of CaCl₂ and 5 μL of a 0.1 M spermidine solution were added to NPs and mixed for 10 minutes at RT. The mix was centrifuged at 5000 rpm (Spectrafuge 16M from Labnet) for 15 seconds at RT, the supernatant was removed and freezer cold 100% ethanol (60 μL) was added to wash the pellet. After another centrifugation step and removal of the supernatant, the coated NPs were resuspended in cold 100% ethanol (5 μL) and loaded in the center of a macrocarrier.

The DNA coated NPs were bombarded onto plant tissues as described in Sanford et al. (1993). A Bio-Rad PDS-1000/He biolistic gun and Bio-Rad biolistic supplies were used. Five different rupture disks (650, 900, 1100, 1350 or 1550 psi) and two different target distances (6 cm or 4 cm) were tested. The typical bombardment parameters used in this study for NPs were 1350 psi, 4 cm target distance and 2 shots. The 4 cm target distance was achieved by placing the sample over a Petri dish with the bottom part upwards on the 6 cm shelf of the gene gun.

For the Treatment #4, the pellet obtained after CaCl₂/Spe coating of NP with mCherry expressing plasmid was washed and pelleted (5000 rpm, 15 seconds) 3 times with ethanol (60 μL) to remove any non-coated free DNA. The resulting pellet was mixed with 2 μL of 0.6 μm gold (30 μg μL⁻¹ in sterile ddH₂O, Bio-Rad cat#165-2262) and 1 μg of a 250 ng/μL stock of GFP expressing plasmid DNA and followed by a second round of DNA coating. For preparing of DNA coated NP and GP mix, c(NP+GP), the NPs were mixed by pipeting with the 2 μL of the 0.6 μm gold suspension. To this mix of particles, 1 μg of a 250 ng/μL stock of DNA plasmid was added and followed the described protocol for DNA coating. Bombardments with those two types of NP-GP mixes were made once at 1100 psi and 6 cm target distance.

Statistical Analysis:

The graphs presented in FIGS. 3 and 4 represent the mean of 2 to 4 repeats±standard error. The comparisons between treatments were done by ANOVA-Duncan Test (α=0.05) using the SAS 9.2 statistical program.

Fluorescence Microscopy:

Fluorescence and bright field images were taken with a 10×A-Plan (numerical aperture, N.A. 0.25) objective of a Zeiss Axiostar plus microscope. For GFP images, an Endow GFP BP filter was used (λₑₓ=470 nm, beam splitter at 495 nm and λₑₘ=525 nm); for mCherry images, a Texas Red filter was used (λₑₓ=560 nm, beam splitter at 595 nm and λₑₘ=645 nm), both from Chroma Technology Corp. Images were taken with a ProgRes C3 digital camera and the ProgRes Capture Pro 2.6 software from Jenoptik, and were edited for publication using Adobe Photoshop software from Adobe Systems Inc.

Differential Interference Contrast (DIC) Microscopy:

DIC and epi-fluorescence images were taken with an upright Nikon Eclipse 80i microscope. A motorized rotary stage (SGSP-60YAM, Sigma Koki) was coupled to the fine-adjustment knob on the microscope to help image sample areas with different depths. For the DIC mode, the samples were illuminated through an oil immersion condenser (N.A. 1.40) and the optical signals were collected with a 100×Plan Apo N.A.1.40 oil immersion objective. One bandpass filter with central wavelength in 700 nm and a full width at half maximum of 13 nm was inserted into the light path in the microscope. For the fluorescence images, a filter cube containing one 480 nm bandpass filter, one 500 nm dichroic mirror and one 530 nm bandpass filter was used. The optical filters were obtained from Semrock. An Andor iXon^{EM}+ camera (512×512 imaging array, 16 μm x16 μm pixel size) and the software ImageJ were used to record and analyze the DIC and fluorescence images.

Transmission Electron Microscopy (TEM), Scanning Electron Microscopy (SEM), and Scanning Transmission Electron Microscopy (STEM) Imaging:

TEM and STEM investigations were done by placing small aliquot of an aqueous suspension on a lacey carbon film coated 400 mesh copper grid and drying it in air. The TEM images were obtained on a Tecnai F² microscope. Particle morphology was determined by SEM using a Hitachi S4700 FE-SEM system with 10 kV accelerating voltage.

### Results

### Improvement of MSN Delivery in Plant Cells by Altering Particle Properties, DNA Coating Procedure and Bombardment Parameters

Increasing the Density of MSN by Gold Plating

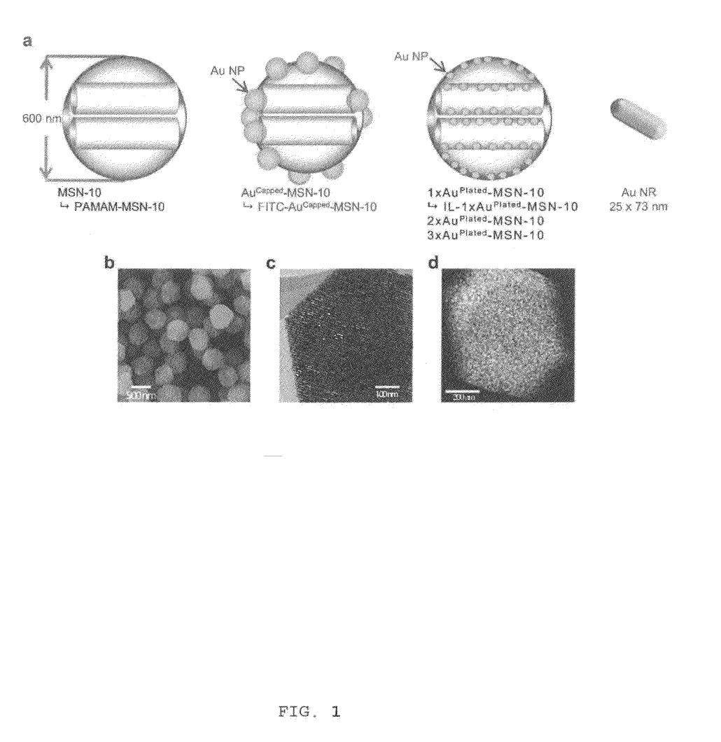

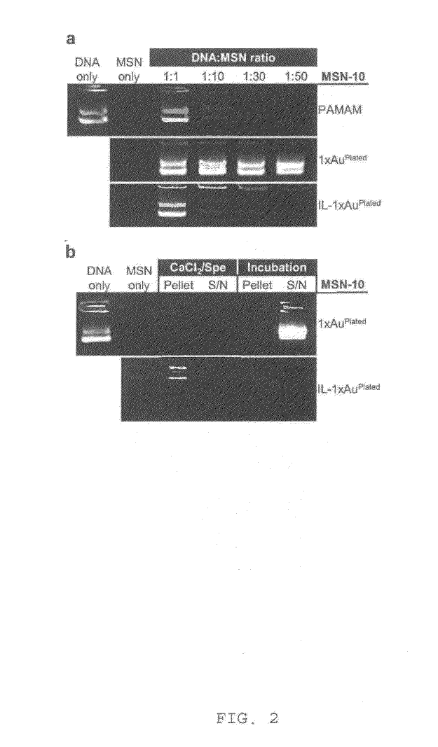

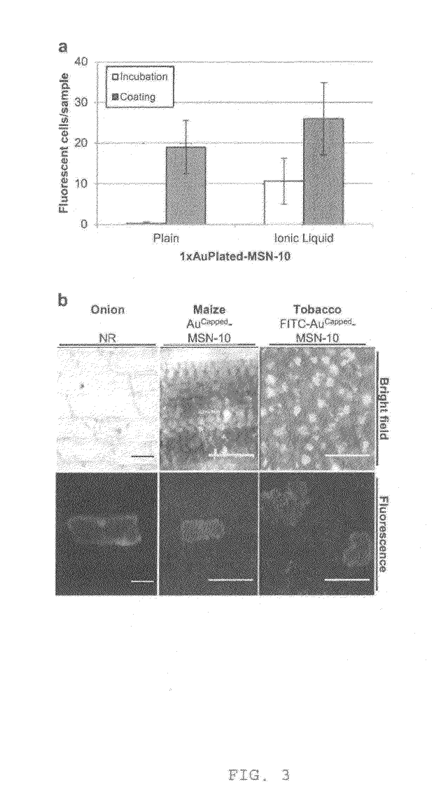

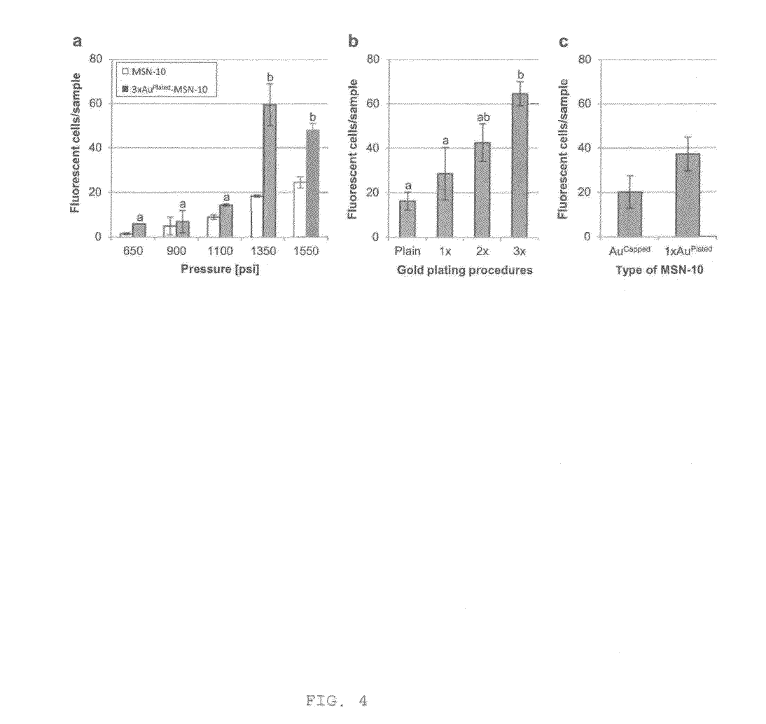

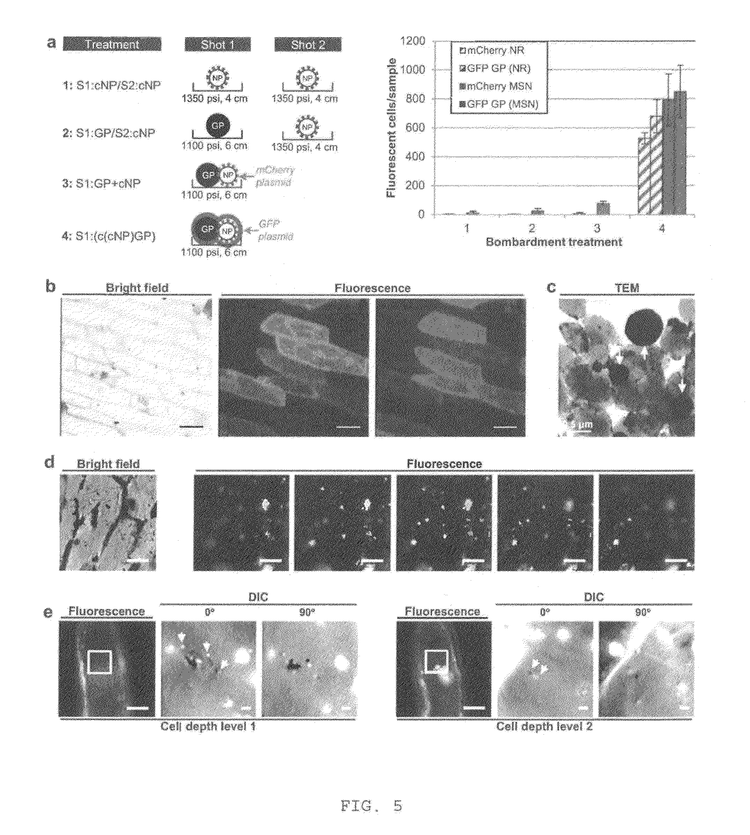

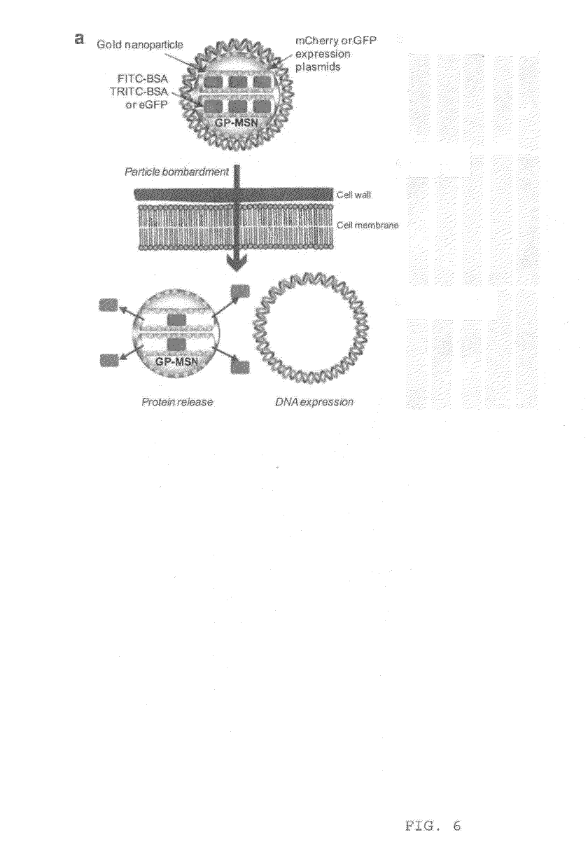

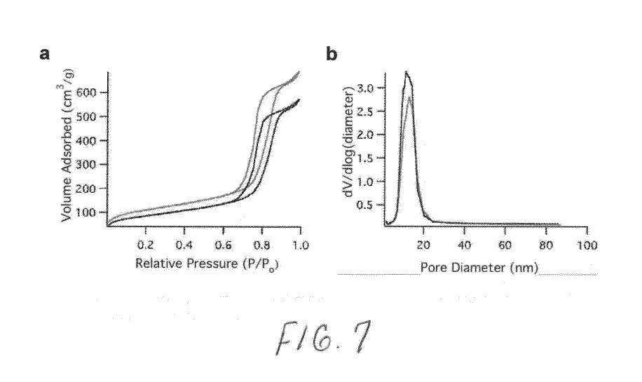

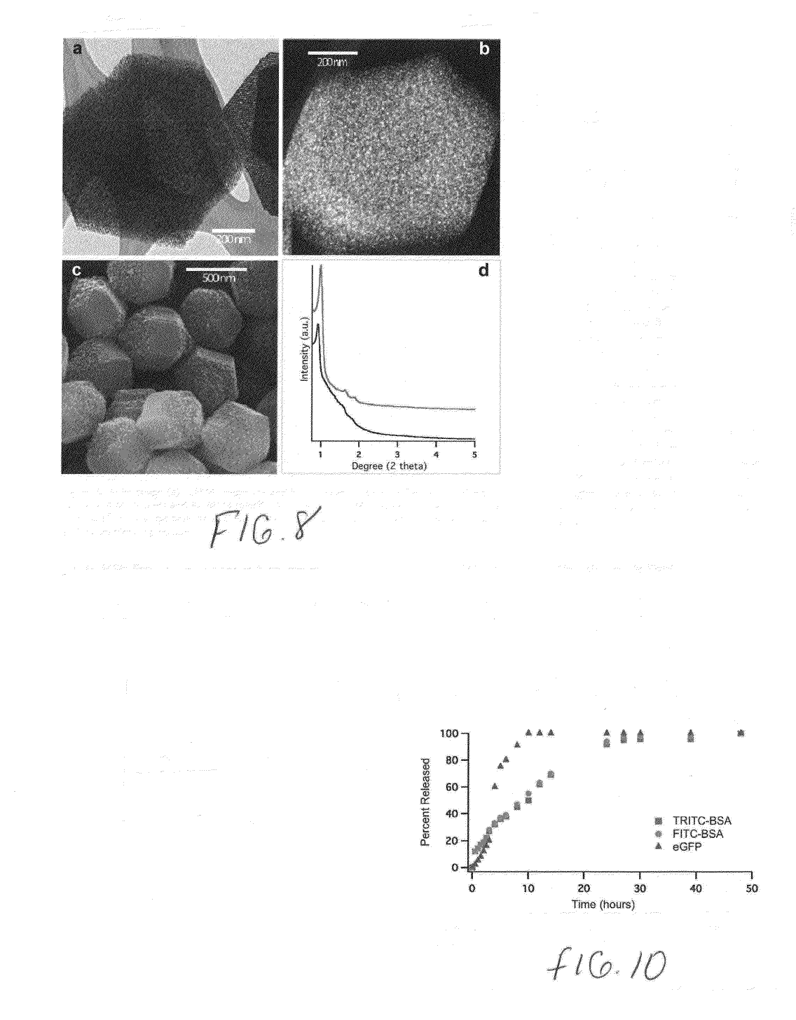

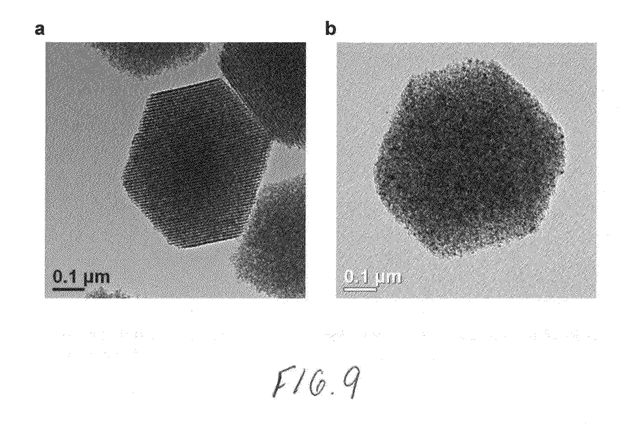

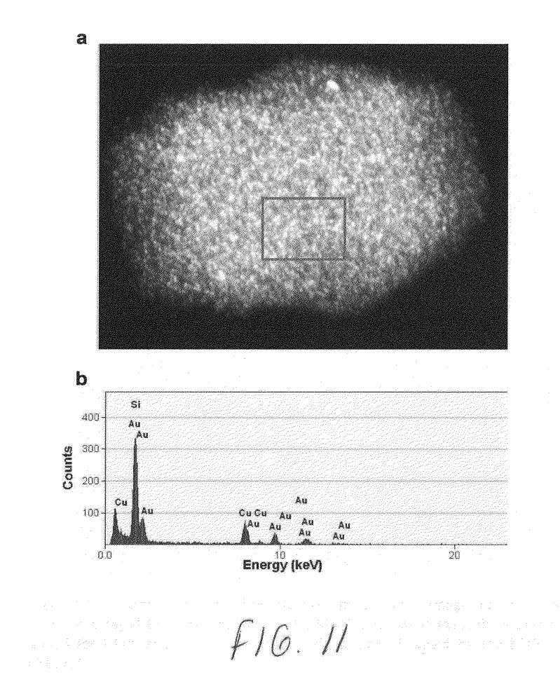

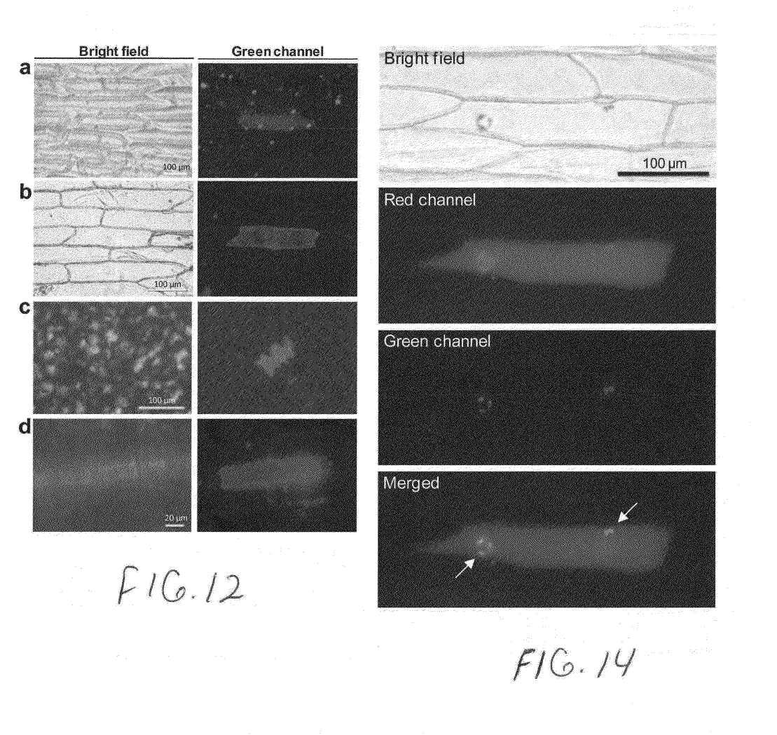

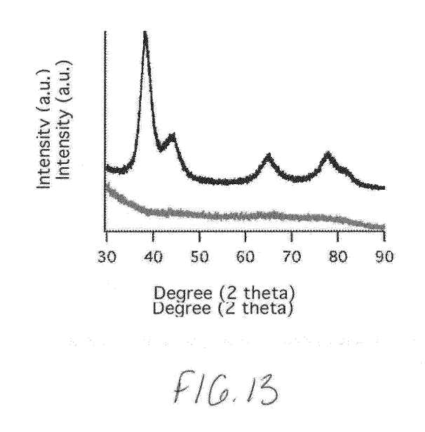

First particle density was modified because density is a major parameter that affects delivery. In this study, MSN with 10 nm pore size (MSN-10) were employed (Kim et al., 2010). These MSN were around 600 nm in diameter (FIG. 1A) but their porous structure and the lower density of the silica material made them much lighter than a gold particle of the same size. A scanning electron micrograph (SEM) can be seen in FIG. 1B.

To increase the density of MSN, two different strategies were designed: (1) capping of the pores of the MSN with gold NPs (FIG. 1A, Au^{Capped}-MSN-10) or (2) gold plating of the MSN surface. The first approach is analogous to one described previously (Torney et al., 2007), which was proven to allow for controlled release of the encapsulated cargo and to increase the performance of the bombardment of plant tissues.

The second approach involved plating gold on the surface of the MSN including the pore walls, a procedure that was repeated multiple times to increase the surface gold loading and, thus, the density of the MSN. In this example, MSN-10 were gold-plated 1, 2 or 3 times resulting in the 1×, 2× or 3×Au^{Plated}-MSN-10 (FIG. 1A), respectively. Transmission electron microscopy (TEM) and scanning transmission electron microscopy (STEM) images of these MSN are presented in FIGS. 1C and 1D. As is observed in FIG. 1D, the gold plating steps produced gold nanoparticles attached to the surfaces of MSN-10, which can be seen as white dots.

The most unique feature of MSN is the high surface area and relatively large pore size. These properties allow for the incorporation of gold via the plating method. As seen in Table 1, the gold plating procedures decreased both the surface area and the pore volume of the MSN, but the final value can be considered sufficient for the encapsulation of molecules. The density of silica is 2.2 g mL⁻¹ and the density of gold is 19.3 g mL⁻¹. Therefore, any amount of gold-plated on the surfaces should increase the density of the MSN. Increasing the density provides the NPs more momentum during the bombardment and is expected to improve the amount of NPs introduced into plant cells. Both the gold capping and gold plating processes involve the use of gold to increase the density, but the gold plating technique seems to allow more gold due to the amount of surface area capable of plating compared to the number of pores that can be capped per MSN. The gold plating method can be considered a more straightforward technique that does not involve the synthesis of gold NPs, attachment to the pore entrances, and subsequent uncapping to release the encapsulated molecules.

TABLE 1
Pore volume and surface area of MSN-10 subjected
to 1x, 2x or 3x gold plating procedures.

| | Surface area (m²/g) | Pore volume (mL/g) |
|---|---|---|
| MSN-10 | 385 | 1.04 |
| 1xAu^{Plated}-MSN-10 | 351 | 0.98 |
| 2xAu^{Plated}-MSN-10 | 318 | 0.89 |
| 3xAu^{Plated}-MSN-10 | 308 | 0.88 |

DNA Coating Procedures onto the Nanoparticles

DNA or RNA delivery to living cells is one of the most important tools in biotechnology. When this delivery is mediated through NPs it usually relies on the ability of their surface to bind electrostatically to the negatively charged nucleic acid molecules. In previous work, the NP-DNA coating was done by simple incubation, namely, DNA and MSN were incubated in water for 2 hours before bombarded into plant cells (Torney et al., 2007). To improve the MSN-DNA binding capability, two surface functionalization methods were tested to provide an overall positive charge on the MSN-10, which as a consequence, is electrostatically attracted to negatively charged DNA.

PAMAM-MSN-10 (FIG. 1A) was surface functionalized with a polyamidoamine dendrimer (PAMAM) layer that improved the MSN-DNA complexation and consequently DNA transfection (Radu et al., 2004). IL-1×Au^{Plated}-MSN-10 (FIG. 1A) was covalently surface functionalized with the ionic liquid (IL), 1-propyl-3-methylimidazolium bromide, to maintain a permanent positive charge on the MSN. The negatively charged nature of MSN-10 was reflected on its negative zeta potential (−28.0 mV), while the surface functionalized PAMAM-MSN-10 and IL-1×Au^{Plated}-MSN-10 had +30.0 and +28.1 mV, respectively. This change in the MSN surface charge led to an improved DNA binding after a one-hour incubation period. The DNA-MSN complexation experiments (FIG. 2A) showed how both functionalized MSN, PAMAM-MSN-10 and IL-1×Au^{Plated}-MSN-10, had a nearly complete DNA complexation in the 1:10 (DNA:MSN) ratio, while 1×Au^{Plated}-MSN-10 did not retain any of the DNA even with 1:50 ratio. This result suggests that both functionalizations have enhanced the DNA binding capacity of the MSN-10 or 1×Au^{Plated}-MSN-10.

To quantitatively test the differences of these NPs and other parameters in plant cells through biolistic transformation, the number of fluorescent cells in onion epidermis tissues was measured one day after they were bombarded with MSN coated with GFP or mCherry expressing plasmid DNA. The tissue can be cut into flat rectangles containing homogeneous shaped cells that facilitate the comparison between replicates and offered excellent fluorescent imaging properties.

As seen in FIG. 3A (white bars, Incubation), under the same gene gun conditions, more cells were transiently expressing mCherry after bombardment with the surface functionalized IL-1×Au^{Plated}-MSN-10 (10.66±5.66) than with the non-functionalized 1×Au^{Plated}-MSN-10 (0.33±0.33). This result demonstrates that the positively charged MSN provides an advantage for DNA expression in living cells than the non-functionalized one, likely due to more DNA coated MSN delivered into plant cells.

In a typical biolistic-mediated plant transformation procedure, DNA molecules are coated onto gold or tungsten microparticles by CaCl₂ and spermidine (CaCl₂/Spe) prior to bombardment (Klein et al., 1987; Sanford et al., 1993). The DNA-MSN incubation method was compared to the CaCl₂/Spe DNA coating protocol (Frame et al., 2000) using 1×Au^{Plated}-MSN-10 (negatively charged surface) and IL-1×Au^{Plated}-MSN-10 (positively charged surface). In the MSN-DNA complexation experiments (FIG. 2B), the amount of DNA and MSN used per shot in a bombardment procedure was tested for both coating protocols. The incubation protocol worked only with the positively charged surface IL-1×Au^{Plated}-MSN-10, while for the negatively charged 1×Au^{Plated}-MSN-10, all the DNA was found free in the supernatant. The CaCl₂/Spe protocol, on the other hand, permitted DNA complexation in both types of MSN, regardless of the charge of their surface (FIG. 2B). For the same gene gun conditions, to bombard onion epidermis cells, the CaCl₂/Spe DNA coating protocol worked significantly better (P=0.0182) than the incubation (FIG. 3A). The CaCl₂/Spe coating protocol allowed for coating onto various NPs and bombardment of different plant tissues. FIG. 3B shows the delivery of either GFP or mCherry expressing plasmids coated onto various types of NPs such as NRs, Au^{Capped}-MSN-10 and FITC-Au^{Capped}-MSN-10 (FIG. 1A), into onion epidermis, maize and tobacco leaves. Two different plasmids were coated simultaneously onto the NPs, which resulted in the co-expression of both GFP and mCherry in same cells (data not shown).

The delivery of DNA or RNA molecules by NPs into plant cells has been reported. In all cases, the DNA-NP coating mixture was incubated for 20 minutes to 12 hours (Torney et al., 2007; Liu et al., 2008; Liu et al., 2009; Pasupathy et al., 2008; Silva et al., 2010; U.S. Publication 2011/0059529). In some cases, adding an L-lysine solution (Liu et al., 2008), an ultrasonication step (Liu et al., 2009) or an amino functionalization to the NP (U.S. Publication 2011/0059529) had to be done to promote DNA-NP complexation. The CaCl₂/Spe DNA coating protocol is efficient in different types of NPs regardless of their ionic nature. Therefore, this coating protocol is used in the rest of the experiments unless otherwise indicated. This procedure may help to reduce the reliance of surface functionalization of NPs in DNA delivery, thus making it easier for the design and manufacture of the NPs.

Parameters Affecting Nanoparticle Delivery to Plant Cells Through Biolistics

To improve the NP delivery efficiency in plant tissues or cells, a number of parameters used in the biolistic system were tested, including target distances and the type of rupture disk. In previous work, 650 psi rupture disks and 10 cm target distance were used for tobacco leaves and maize immature embryos (Torney et al., 2007). Bombardment of onion epidermis tissue with Au^{Capped}-MSN-10 showed that the rupture disks for higher pressures (1350 or 1550 psi) and smaller target distances (4 cm) resulted in an improved transient expression (data not shown). Twice bombarded samples had more cells transiently expressing the fluorescent proteins than the cells bombarded only once, which is in agreement with an earlier publication (Klein et al., 1988). Therefore, all DNA-NP delivery data presented below utilized the repeat bombardment protocol for each sample unless otherwise indicated.

FIG. 4A shows the data comparing MSN-10 and the gold-plated 3×Au^{Plated}-MSN-10 (FIG. 1A), using 5 different rupture disks. As can be seen, the number of fluorescent plant cells bombarded with MSN-10 and 3×Au^{Plated}-MSN-10 did not differ much when using rupture disk types 650, 900 and 1100 psi, respectively. However, the number of fluorescent cells after bombardment with 3×Au^{Platted}-MSN-10 increased significantly when using rupture disk types 1350 and 1550 psi (P=0.0017). These data demonstrated that the increase in the density acquired during the gold plating of 3×Au^{Plated}-MSN-10 improved its performance comparing to the MSN-10 when higher pressures are used.

The delivery of four different types of gold-plated MSN-10 (MSN-10, 1×, 2× and 3×Au^{Plated}-MSN-10) was compared in bombarded onion epidermis cells under the same gene gun conditions (1350 psi rupture disk and 4 cm target distance). As shown in FIG. 4B, each time MSN-10 went through a gold plating process, the increase in density enhanced its delivery to plant cells, which can be indirectly measured by the increasing number of cells expressing mCherry. The optimal MSN for DNA delivery was 3×Au^{Plated}-MSN-10, as was determined by the significantly greater number of fluorescent cells (P=0.015) than MSN-10 or 1× or 2×Au^{Plated}-MSN-10 (FIG. 4B).

Thus, the gold plating technique for treating MSN enhances the performance of delivery to plant cells by the biolistic method. The 1×Au^{Plated}-MSN-10 also showed better performance than the Au^{Capped}-MSN-10 (FIG. 4C). Compared to the Au^{Capped}-MSN, the Au^{Plated}-MSN is easier to manufacture and allows for more functionalization. For example, gold nanoparticles alone may be used as caps for the Au^{Capped}-MSN (Trewyn et al., 2007), while any “hard” or “soft” cap may be used with the Au^{Plated}-MSN. This allows us more freedom to design a delivery system tailored around the cell type and cargo of interest. While each gold-plating process increases the density of MSN, it also decreases surface area and pore volume proportionally (Table 1).

Co-Bombardment of Nanoparticles with Solid 0.6 μm Gold Microparticles to Enhance Efficient Delivery Through the Biolistic Method

In an attempt to extend the biolistic delivery system for plant cells to different types of NP, commercially available 0.6 μm gold particles (GP), a standard microcarrier for delivering DNA in biolistic-mediated plant transformation, were used as a microcarrier for various NPs. It was hypothesized that the NPs would attach to the GP in the presence of DNA molecules and/or chemicals such as CaCl₂ and spermidine. This type of NP-GP-DNA complex would more readily penetrate plant tissues through bombardment.

Using two types of NPs different in size and nature, Au^{Capped}-MSN-10 or gold NRs (FIG. 1A), four different treatments were tested involving different NP, GP, and DNA combinations and delivery strategies. FIG. 5A summarizes the four treatments and results. In Treatment #1 (FIG. 5A, S1:cNP/S2:cNP), plant samples were bombarded twice with either DNA-coated NRs (mCherry NR) or MSN (mCherry MSN). This treatment yielded 4±3 (NR) or 12±11 (MSN) fluorescent cells per sample on average as was typically observed in this study. In Treatment #2 (FIG. 5A, S1:GP/S2:cNP), the samples were first bombarded with the GP followed by a second shot with the DNA coated NPs. This was to test whether the holes on the cell walls made by the GPs would ease the following NP introduction. However, this treatment did not enhance the delivery of NP-DNA into plant cells as indicated by the number of fluorescent cells (4±1 and 27±15 for NR and MSN, respectively).

In Treatment #3 (FIG. 5A, S1:GP+cNP), GP was first loaded onto the macrocarrier followed by the loading of DNA-coated NPs, and plant tissues were bombarded once. In this treatment, a slight increase of red fluorescent cells (76±15) can be observed in samples bombarded with GP+MSN complex (mCherry MSN). The results of Treatment #2 and #3 suggested that the delivery of MSN, not nanorods, could be enhanced by the mixture of DNA-coated NPs and GP on the macrocarrier.

In Treatment #4 (FIG. 5A, S1:c(cNP)GP)), the mCherry expressing plasmid DNA was first coated onto the NPs and then the coated NPs were washed 3 times with ethanol to get rid of any free DNA molecules. Then GP and a GFP expressing plasmid DNA were added for a second round of the CaCl₂/Spe coating procedure. Plant tissues were bombarded once using this c(cNP)GP complex. As can be seen in the graph of FIG. 5A, this treatment led to a drastic improvement of NP delivery, indirectly indicated by the expression of both mCherry for NP delivery (mCherry NR or MSN) and GFP for GP delivery (GFP GP(NR) or (MSN)). This treatment resulted in around 130 times better for NR delivery and over 60 times in the case of MSN. Both red and green fluorescent proteins were expressed in 77% (NR) or 93% (MSN) of the cells, indicating the co-delivery of both GP and NPs (FIG. 5B). In both MSN and NR delivery experiments, the number of green fluorescent cells was slightly higher than the red fluorescent cells. This may suggest that more GP than NP were delivered by this procedure. It was confirmed that a single DNA coating procedure for a mixture of GP and NP, c(GP+NP), was also effective for co-bombardment (data not shown).

The c(GP+MSN) complex was examined under TEM. FIG. 5C shows an example of a heterogeneous population of MSNs and GPs (white arrows). While this delivery strategy has been efficient and reproducible with onion epidermal tissues, this type of particle agglomeration can cause excessive damage to plant tissues upon bombardment. Therefore, different types of plant tissues may have different ratios of NPs and GP and biolistic gun parameters.

Further evidence for the presence of NPs inside plant cells using GP as a carrier were collected by performing optical sectioning of the sample with either fluorescence microscopy or differential interference contrast (DIC) microscopy with the aid of a high precision motorized rotary stage. As shown in FIG. 5D, after the c(GP+FITC-Au^{Capped} MSN-10) complex bombardment, the FITC labeled MSNs were found to be distributed in different axial planes of the bombarded onion epidermis cells, confirming the introduction of multiple MSNs inside the tissue. After bombardment with the c(GP+NR) complex, NR detection was used to examine mCherry expressing onion cells. Multiple NRs (white arrows in FIG. 5E) could be identified based on the change of the DIC image patterns at 0° and 90°: after rotating 90°, the DIC images of NRs changed from dark to bright or from bright to dark; while the DIC images of other cellular organelles did not have such an effect (Wang et al., 2010; Stender et al., 2010). In addition, NRs were detected inside a red fluorescent cell at two planes located at different depths which confirms that this method can deliver multiple NRs and DNA into the same cell. This strategy also worked in different NP delivery in other plant explants like tobacco or maize leaf tissues (data not shown).

Using this co-bombardment strategy, two different types of NPs, MSNs and NRs were effectively introduced into plant cells. This suggests that the strategy may be applicable to NPs of different sizes, shapes, and properties. In mammalian cell systems, the use of a complex formed by the mixture of DNA, NPs and microparticles has been reported (Svarovsky et al., 2008). In this case, a complex of plasmid DNA, surface functionalized 36 nm gold NPs and 1.5 μm gold microparticles was formed by electrostatic attachment. Their goal was to deliver large amounts of DNA to mouse NIH 3T3 fibroblast cells and ear tissue by biolistics. In the present case, the particles were not subjected to any surface functionalization, smaller (0.6 μm) microparticles were used, and only 1 μg of DNA was coated using the CaCl₂/Spe protocol.

### Conclusions

In this study, three methods were demonstrated to improve the introduction of nanoparticles and DNA into plant cells through the biolistic system. Firstly, the gold plating of MSN increases the density and performance in biolistic mediated delivery. This improvement allows the introduction of the MSNs into plant cells more efficiently. Even though this gold plating technique may diminish the porosity of MSN and, as a result, the cargo capacity, it overcomes the disadvantages of bombarding plant tissues with MSNs or other types of NP, when applicable, due to their smaller size and density.

Secondly, the CaCl₂/Spe DNA coating protocol, routinely used in gold or tungsten microparticles in plant transformation, is suitable for the NPs employed herein. Using the CaCl₂/Spe coating method, instead of DNA-NP incubation protocol, NP-mediated DNA delivery efficiency can be improved. This coating method is applicable to different kinds of NP(NR and MSN) regardless of their surface ionic state. Therefore, this method could reduce the burden needed for surface functionalization steps designed to improve DNA binding capacity.

Finally, the complex formed by NPs, GPs and DNA after performing the CaCl₂/Spe coating protocol significantly enhances the introduction of NPs to plant cells through bombardment. For each particular case, the NP/GP ratio should be tested to balance the possible mechanical damage to plant cells upon bombardment.

### Example 2

### Materials and Methods

### Preparation of MSN-10.

To synthesize MSN with 10 nm pore size (MSN-10), P104 surfactant (7.0 g) was dissolved in HCl (273.0 g, 1.6 M) and stirred (1 hour at 55° C.), followed by rapid addition of tetramethyl orthosilicate (TMOS) (10.64 g). The solution was stirred for 24 hours, transferred to a high-pressure vessel and placed in an oven at 150° C. for 24 hours. The product was filtered and washed with water and methanol. The surfactant was removed by heating the material to 550° C. for 6 hours.

### Preparation of Au-MSN.

To synthesize Au(en)₂Cl₃ for gold modification, ethylenediamine (0.45 mL) was added to an aqueous solution of HAuCl₄.3H₂O (1.0 g) in water (10 mL) and stirred for 30 minutes. Ethanol (70 mL) was added and the Au(en)₂Cl₃ precipitate was filtered, washed with ethanol and dried under vacuum. Three cycles of gold functionalization were performed, and for each cycle, Au(en)₂Cl₃ (0.372 g) was dissolved in water (150 mL) and the pH adjusted to 10.0 using NaOH. After adding MSN-10 (2.0 g), pH was readjusted to 9.0 with NaOH and stirred for 2 hours. The final product (Au-MSN) was filtered and dried under vacuum for 2 days and then, reduced under H₂ flow (150° C., 3 hours).

Fluorescent Labeling of Au-MSN.

For FITC labeling, FITC (5 mg, 12.8 μmol) was added to 3-aminopropyltrimethoxysilane (APTMS) (13 μmol) in dry DMSO (0.5 mL), stirred for 30 minutes and then grafted on to Au-MSN (1.0 g) in toluene. The suspension was refluxed for 20 hours under nitrogen and the resulting material was filtered, washed with toluene and methanol, and dried under vacuum overnight.

MSN Surface Area and Porosity Measurement.

The surface area and average pore diameter measurements were recorded using nitrogen sorption analysis in a Micromeritics ASAP 2020 BET surface analyzer system. The Brunauer-Emmitt-Teller (BET) and the Barrett-Joyner-Halenda (BJH) equations were used to calculate apparent surface area and pore size distributions, respectively, of MSN samples. Degas of MSN samples were done at 100° C. overnight before analysis.

Zeta Potential Measurements.

MSN samples (1 mg) were sonicated in phosphate buffered saline (PBS) pH 7.4 (10 mL), 10 mM NaCl for 5 minutes and then analyzed on a Zetasizer (Malvern Instruments). The reported zeta potential value is an average of 10 individual measurements. For DNA coated MSN measurements, Au-MSN (1 mg) were coated with ER-rk (Nelson et al., 2007) plasmid (10 μg) and the sample was vigorously shaken to suspend the MSN in the buffer.

Transmission Electron Microscopy (TEM), Scanning Electron Microscopy (SEM), and Scanning Transmission Electron Microscopy (STEM) Imaging.

TEM and STEM investigations were done by placing small aliquot of an aqueous suspension on a lacey carbon film coated 400 mesh copper grid and drying it in air. The TEM images were obtained on a Tecnai F² microscope. Particle morphology was determined by SEM using a Hitachi S4700 FE-SEM system with 10 kV accelerating voltage.

Protein Loading and In Vitro Release.

For protein loading, Au-MSN (20 mg) were sonicated in PBS (5 mL) solution (pH 7.4) followed by the addition of FITC-BSA or TRITC-BSA (Sigma-Aldrich) (15.7 mg). For eGFP (BioVision) encapsulation, Au-MSN (2 mg) and 0.6 mg of the protein were used. The protein-Au-MSN mixture was stirred at room temperature (22° C.) for 24 hours and then centrifuged. The supernatant was removed and the remaining MSNs were resuspended briefly in PBS solution (pH 7.4) and lyophilized. To determine protein loading, the fluorescence emission of the FITC-BSA or eGFP in the supernatant was measured using a spectrophotometer. The measurement indicated that the loaded protein was 0.625 and 0.15 mg of protein per mg of GP-MSN for FITC-BSA and eGFP, respectively (Table 2). To measure protein release from the Au-MSN, protein-loaded Au-MSN was stirred in PBS (pH 7.4) (2 mL) solution for a period of time. An aliquot was removed and centrifuged to separate the released protein in the supernatant from the MSNs in the pellet, and the fluorescence intensity was measured using at a spectrophotometer (FITC-BSA or eGFP: λ_{Ex}=488, λ_{Em}=518 nm; TRITC-BSA: λ_{Ex}=557, λ_{Em}=576 nm).

Plant Materials.

White onion epidermis tissue rectangles (2×3.5 cm) were placed in dishes containing agar media (0.5 mM 2-(N-morpholino)-ethanesulfonic acid (MES), and 15 g L⁻¹ of BD Bacto agar, pH 5.7), facing the peeled surface upwards. For tobacco and teosinte leaf bombardment, leaves from 6 to 8 week old in vitro-grown tobacco plants (Nicotiana tabacum var. Petite Havana) and leaf pieces of 2-month old teosinte plants (Ames 21785, USDA/ARS/North Central Regional Plant Introduction Station, Iowa State University) were placed with the adaxial surface up on agar media.

Biolistic Method.

For the delivery of protein filled Au-MSN, freshly prepared Au-MSN suspensions (5 μL, 20 μg μL⁻¹) in ethanol were loaded onto a macrocarrier. Using a PDS-1000/He biolistic gene gun (BioRad Laboratories), plant samples were bombarded twice at 1350 psi rupture disks and 6 cm target distance. For the delivery of plasmid DNA coated, protein filled Au-MSN, 4 μL of DNA (250 ng μL⁻¹) was added to 10 μL of protein filled Au-MSN (10 μg μL⁻¹ stock, freshly prepared in ddH₂O) to make a final ratio of 1 μg DNA to 100 μg Au-MSN per shot. DNA precipitation onto Au-MSN was achieved by adding 12.5 μL of 2.5 M CaCl₂ (1 M final concentration) and 5 μL of 0.1 M spermidine (16 mM final concentration) to the DNA/Au-MSN mixture. After mixing the contents, the mixture was briefly centrifuged for 15 seconds (5000 rpm, room temperature). The supernatant was discarded, the pellet was washed with cold 100% ethanol (60 μL) and centrifuged again. After removal of the supernatant, DNA-coated protein-loaded Au-MSNs were resuspended in cold 100% ethanol (5 μL) and loaded in a macrocarrier. Each plant sample was bombarded twice at 1350 psi and 6 cm target distance.

Fluorescence Microscopy Imaging.

Bright field and fluorescence images were taken with 10×A-Plan and or 40×A-Plan objectives of a Zeiss Axiostar plus microscope with a green channel (Endow GFP BP: λₑₓ=470 nm, beam splitter=495 nm and λₑₘ=525 nm) and a red channel (Texas Red: λₑₓ=560 nm, beam splitter=595 nm and λₑₘ=645 nm) filters (Chroma Technology Corp.) were used. Microscopy images were taken using ProgRes Capture Pro 2.6 software and a ProgRes C3 digital camera, both from Jenoptik. If necessary, images were edited using Adobe Photoshop software (Adobe Systems Inc).

### Results and Discussion

MSN Material Synthesis and Characterization

The 10 nm pore-sized and gold-plated MSN (Au-MSN) and Au-MSN-mediated protein and DNA co-delivery in plants is illustrated in FIG. 6. The synthesis of Au-MSN material was done according to the protocol described in Example 1. Briefly, for the synthesis of MSN with 10-nm pore size (MSN-10), 7.0 g of P104 surfactant was dissolved in 273.0 g of 1.6 M HCl and stirred (1 hour at 55° C.), followed by rapid addition of 10.64 g of tetramethyl orthosilicate (TMOS). The solution was stirred for 24 hours, transferred to a high-pressure vessel and placed in an oven at 150° C. for 24 hours. The product was filtered and washed with water and methanol. The surfactant was removed by heating the material to 550° C. for 6 hours. To synthesize Au(en)₂Cl₃ for gold modification, 0.45 mL of ethylenediamine was added to an aqueous solution of 1.0 g of HAuCl₄.3H₂O in 10 mL of water and stirred for 30 minutes. Seventy mL of ethanol was added and the Au(en)₂Cl₃ precipitate was filtered, washed with ethanol and dried under vacuum. Three cycles of gold plating were performed, and for each cycle, 0.372 g Au(en)₂Cl₃ was dissolved in 150 mL of water and the pH adjusted to 10.0 using NaOH. After adding 2.0 g of MSN-10, pH was readjusted to 9.0 with NaOH and stirred for 2 hours. The product was filtered and dried under vacuum for 2 days and then reduced under H₂ flow (150° C., 3 hours).

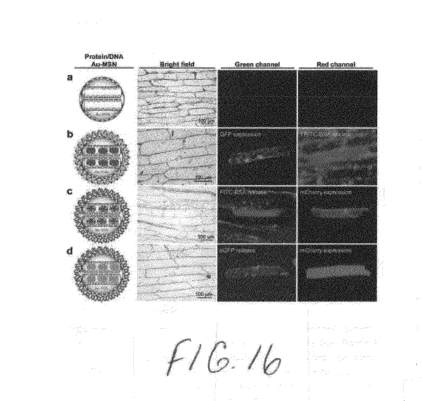

Nitrogen sorption analyses, electron microscopy measurements, and powder X-ray diffraction (XRD) spectroscopy were utilized to fully characterize the Au-MSN materials that were synthesized by repeated gold reduction on the surface of the MSN. The repeated surface gold reduction was necessary to increase the nanoparticle density for successful delivery to plant cells by bombardment (Martin-Ortigosa et al., 2012). The Au-MSN exhibited a type IV isotherm with a BET surface area of 313 m²g⁻¹ and a pore volume of 0.89 cm³g⁻¹ and the BJH pore size distribution measurement showed a negligible decrease in pore diameter after three cycles of gold reduction (FIG. 7). As is observed in the transmission electron microscopy (TEM) image (FIG. 8A and FIG. 9), the pore channels can be seen as parallel stripes running the length of the MSN, confirming the XRD pattern of a well ordered material. The scanning transmission electron microscopy (STEM) image (FIG. 8B and FIG. 11A) shows the presence of gold nanoparticles on the surface of Au-MSN after the repeated deposition and reduction of gold salt. Scanning electron microscopy (SEM) image shows that the structure, shape and size of Au-MSNs are around 600 nm in diameter and have consistent particle size and morphology (FIG. 8C). Energy dispersive X-ray analysis confirms that presence of gold on the surface of the MSN (FIG. 11B). The X-ray diffraction pattern of the Au-MSN material indicates a well-ordered pore structure characteristic of 2D hexagonal MSN (FIG. 8D). High angle XRD patterns of MSN and Au-MSN confirm the presence of crystalline gold on the MSN (FIG. 13). The overall surface charge of each sample was measured in pH 7.4 PBS. The zeta potential of the MSN-10 (−29.0 mV) decreased slightly after gold was reduced on the surface to form Au-MSN (−25.5 mV). Measuring the zeta potential of plasmid DNA coated Au-MSN proved to be difficult due to significant particle aggregation during the surface charge measurement acquisition. To verify the synthesis of MSN-10 is consistent and the particle morphology and size is conserved, SEMs were recorded from four different batches. These SEMs are included in the supporting information (FIG. 15).

Au-MSN Protein Loading and In Vitro Release

Two different proteins were chosen for Au-MSN loading (Table 2): BSA, fluorescein isothiocyanate (FITC-BSA) labeled or tetramethyl rhodamine isothiocyanate (TRITC-BSA) labeled and eGFP. The size of these proteins (hydrodynamic radius of 4.5 and 2.3 nm for BSA and eGFP, respectively) (Böhme et al., 2007; Hink et al., 2000) was smaller than the 10 nm diameter pore size of the Au-MSN. Therefore, high protein loading into the pores was expected (Katiyar et al., 2005). The amount of each protein that was entrapped in the mesopores was determined by measuring the difference in protein concentration in the supernatant before and after the loading procedure. The measurements indicated that the maximum protein loading, at the conditions studied, was 625 and 150 mg of protein per 1.0 g of Au-MSN for FITC-BSA and eGFP, respectively (Table 2).

TABLE 2
Protein and protein loaded Au-MSN characteristics.

| | | | | Protein/ | |
|---|---|---|---|---|---|
| | Size | Rₕ^{a)} | | Au-MSN | % protein |
| Protein | [kDa] | [nm] | pl | [mg g⁻¹] | released |
| BSA | 66.8 | 4.5 | 4.7 | 625 | 28 |
| GFP | 28 | 2.3 | 6.2 | 150 | 8 |

^{a)}Rₕ is the hydrodynamic radius (Böhme et al., 2007; Hink et al., 2000).

After the proteins were loaded in the Au-MSN, a time course of in vitro release of the loaded proteins was performed at room temperature during which the structure and activity of the proteins were maintained as is evident by continued fluorescence of the released eGFP (Ward et al., 1982). The fluorescently-labeled BSAs showed a continuous release pattern during the first 20 hours, while the eGFP achieved maximum release after 10 hours (FIG. 10). The difference in release kinetics could be attributed to the variation in the amount of protein loaded in the Au-MSN, the difference in protein-pore wall interaction, and the difference in the sizes of eGFP and BSA (Table 2). After 48 hours incubating at room temperature (22° C.) in static conditions in phosphate buffered saline (PBS) solution (pH 7.4), the total percent of protein released was 28% and 8% for BSA and eGFP, respectively. Improving and controlling the percentage of protein release are research activities currently in progress. Subsequent suspension of the protein loaded Au-MSN pellets for further protein release did not yield more fluorescence in the supernatant, suggesting that no more detectable free proteins were released from the MSN (data not shown).

Au-MSN Mediated Protein Delivery to Plant Cells

To introduce protein-encapsulated Au-MSN to plant cells, the biolistic delivery method was employed (Torney et al., 2007; Martin-Ortigosa et al., 2012). Release of FITC-BSA was observed in bombarded onion epidermis cells as early as 30 minutes after bombardment (FIG. 4a). Nevertheless, protein release was typically observed 1 day after bombardment as in the case of eGFP (FIG. 4 b). In general, fluorescently-labeled BSA release was more distinguishable and more frequent than eGFP detection. A typical bombardment for fluorescently labeled BSA release showed hundreds of fluorescent onion epidermis cells, while eGFP release occurred in less than 10 cells per bombarded sample (2 cm×3.5 cm in size). This difference could be due to the smaller amount of protein encapsulated into Au-MSN, the lower release percentage (Table 2) and the overall lower fluorescence emission of eGFP comparing to FITC-BSA (Bale et al., 2010). Although limited eGFP release could be observed in a small number of cells one day after the bombardment, longer periods of time (up to 6 days) were needed to obtain more fluorescent cells, likely due to continued release of eGFP from Au-MSN in plant cells over time.

To prove this system is applicable in other plant tissues, leaves of tobacco and teosinte plants were bombarded as described for onion epidermis tissue. In both cases (FIGS. 12C-D, respectively) cells showing FITC-BSA release were found in the plant tissues one day after bombardment. In the plant tissues tested, the fluorescence was observed throughout the cell, not localized in cell compartments, indicating that the fluorescent proteins were released into and diffused throughout the cell cytoplasm (FIGS. 12A-D).

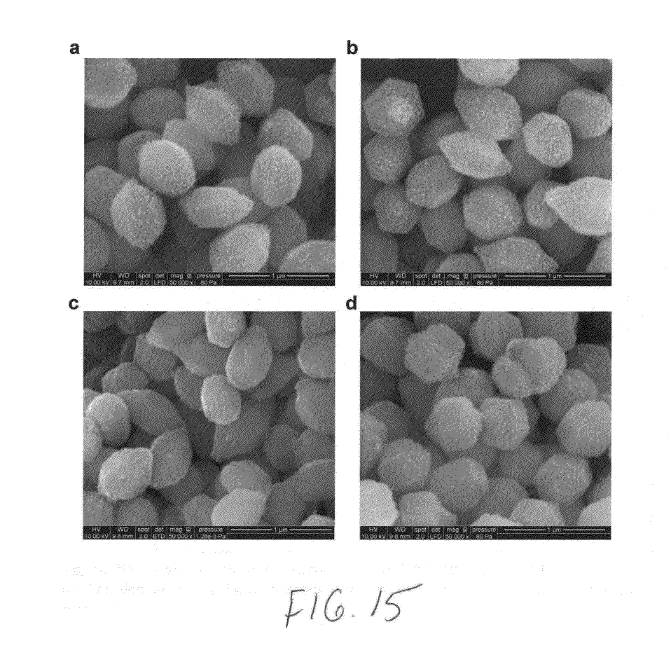

To confirm that the observed cellular fluorescence is due to MSN introduction and not to isolated protein aggregates formed during MSN-loading, Au-MSN were covalently labeled with FITC prior to TRITC-BSA protein encapsulation. Onion epidermis cells bombarded with this material showed red fluorescent cells (due to TRITC-BSA release) and green fluorescent dots (FITC-labeled Au-MSN), confirming that the TRITC-BSA release is associated with the presence of Au-MSN inside the same plant cell (FIG. 14).

Au-MSN Mediated Codelivery of Protein and Plasmid DNA to Plant Cells

Simultaneous delivery of plasmid DNA and protein in onion epidermis cells is shown in FIG. 16. For DNA coating and delivery of protein-encapsulated Au-MSN, an optimized biolistic procedure was employed for Au-MSN (Martin-Ortigosa et al., 2012). The plasmids used were ER-rk (Nelson et al., 2007) (red fluorescent protein mCherry gene expression) when FITC-BSA or eGFP loaded Au-MSN were used, and pLMNC95 (Mankin et al., 2001) (GFP gene expression) for TRITC-BSA loaded Au-MSN. The co-localization of both red and green fluorescent emissions is expected when the codelivery and release of both protein and plasmid DNA is successful.

The control experiment (FIG. 16A) bombarded with empty, non-DNA coated Au-MSN showed no fluorescence on both green and red channels, as expected. Onion tissue bombarded with Au-MSN loaded with TRITC-BSA and coated with the GFP expression plasmid DNA pLMNC95 showed cells simultaneously fluorescent in red (protein release) and in green (DNA expression) (FIG. 10B). Cells bombarded with Au-MSN that was loaded with FITC-BSA and coated with mCherry plasmid DNA showed green fluorescence due to the protein release and red fluorescence due to the DNA expression (FIG. 10C). Finally, when eGFP-loaded and mCherry plasmid DNA coated Au-MSN was bombarded into onion tissue, co-localization of both green fluorescent (eGFP release) and red fluorescent (mCherry gene expression) could be detected (FIG. 10D), indicating the consistency of the system for the codelivery of both biomolecules. The presence of green fluorescence diffused throughout the entire cell in FIG. 10D indicates that the eGFP delivered and released in plant cells remain in the proper configuration. If eGFP denatured or unfolded, then the protein would no longer be fluorescent (Ward et al., 1982).

### CONCLUSIONS

10 nm pore-sized, gold functionalized MSN can be used to load proteins with a hydrodynamic diameter as large as 4.5 nm and release them under physiological conditions. The protein-loaded Au-MSN can be subsequently coated with plasmid DNA and introduced into plant tissues through particle bombardment. The delivery and release of two types of biomolecules, protein and plasmid DNA, can be detected in the same plant cells. Further improvements are currently under way to improve protein encapsulation and release efficiencies of MSN materials as well as frequencies for the biolistic delivery in plant tissues. We anticipate that this novel DNA/protein delivery system will lead to advancements in plant cell and plant genomic manipulation applications and research.

### REFERENCES

Bale et al., ACS Nano, 4:1493 (2010).
Batzer et al., Nucleic Acid Res., 19:5081 (1991).
Bhattacharyya et al., J. Phys. Chem. C, 114:19928 (2010).
Birbaum et al., Environ. Sci. Technol., 44:8718 (2010).
Böhme et al., Chem. Phys. Lett., 435:342 (2007).
Boutileir et al., Plant Cell, 14:1737 (2002).
Casamitjana-Martinez et al., Curr. Biol., 13:1435 (2003).
Chang et al., New Phytologist, 174:46 (2007).
Chen et al., Small, 6:612 (2010).
Chugh et al., FEBS J., 275:2403 (2008).
Cifuentes et al., J. Nanobiotechnol., 8:26 (2010).
Clark et al., Cell, 89:575 (1997).
Clark et al., Microchim. Acta, 131:121 (1999).
Cock & McCormick, Plant Physiol., 126:939 (2001).
Corredor et al., BMC Plant Biol., 9:45 (2009).
Eichert et al., Physiol. Plant., 134:151 (2008).
Elliott et al., Plant Cell, 8:155 (1996).
Etxeberria et al., Plant Signal Behav., 1:196 (2006).
Fletcher et al., Science, 283:1911 (1999).
Fletcher, Dev., 128:1323 (2001).
Frame et al., In Vitro Cell. Dev. Biol. Plant, 36:21 (2000).
Giri et al., Angew. Chem. Int. Ed., 44:5038 (2005).
González-Melendi et al., Ann. Bot., 101:187 (2008).
Grichko et al., NanoBioTechnol., 2:37 (2006).
Hink et al., J. Biol. Chem., 275:17556 (2000).
Ho et al., J. Chem. Technol. Biotechnol., 83:351 (2008).
Hobe et al., Dev. Genes Evol., 213:371 (2003).
Ishiguro et al., EMBO J., 21:898 (2002).
Jackson et al., Dev., 120:405 (1994).
Jeong et al., Plant Cell, 11:1925 (1999).
Jonak et al., Curr. Opin. Plant Biol., 5:415 (2002).
Jung et al., Chem. Commun., 5003 (2009).
Kam et al., J. Am. Chem. Soc., 127:6021 (2005).
Katiyar et al., Microporous Mesoporous Mater., 80:311 (2005).
Kaya et al., Cell, 104:131 (2001).
Khodakovskaya et al., Proc. Natl. Acad. Sci. U.S.A., 108:1028 (2011).
Kim et al., ACS Nano, 5:360 (2010).
Kim et al., J. Nanosci. Nanotechnol., 10:3467 (2010).
Klein et al., Nature, 327:70 (1987).
Klein et al., Proc. Natl. Acad. Sci. U.S.A., 85:4305 (1988).
Klein, in Plant transformation technologies, Wiley-Blackwell, 53 (2011).
Klucher et al., Plant Cell, 8:137 (1996).
Lee et al., Angew. Chem., Int. Ed., 122:2606 (2010).
Lee et al., Biomater., 29:742 (2008).
Li et al., Biomaterials, 32:9546 (2011).
Lim et al., Biomaterials, 30:1197 (2009).
Lin et al., Small, 5:1128 (2009).
Lincoln et al., Plant Cell, 6:1859 (1994).
Liu et al., J. Cent. S. Univ. Technol., 15:768 (2008).
Liu et al., J. Cent. S. Univ. Technol., 9:1007 (2008).
Liu et al., Nano Lett., 9:1007 (2009).
Long et al., Nature, 379:66 (1996).
Lotan et al., Cell, 93:1195 (1998).
Lu et al., J. Agric. Food Chem., 58:2288 (2010).
Lucas et al., ACS Appl. Mater. Interfaces, 2:2198 (2010).
Lynn et al., Dev., 126:469 (1999).
Ma et al., Sci. Total Environ., 408:3053 (2010).
Mankin et al., Plant Mol. Biol. Reporter, 19:13 (2001).
Martin-Ortigosa et al., Small, 8:413 (2012).
Mayer et al., Cell, 95:805 (1998).
Murashige et al., Physiol. Plant., 15:473 (1962).
Nair et al., Plant Sci., 179:154 (2010).
Nelson et al., Plant J., 51:1126 (2007).
O'Farrell et al., Int. J. Nanomedicine, 1:451 (2006).
Ogas et al., Proc. Natl. Acad. Sci. USA, 96:13839 (1999).
Ohad et al., Plant Cell, 11:407 (1999).
Ohtsuka et al., J. Biol. Chem., 260:2605 (1985).
Onelli et al., J. Exp. Bot., 59:3051 (2008).
Pasupathy et al., Biotechnol. J., 3:1078 (2008).
Perez-Donoso et al., Plant Physiol., 152:1748 (2010).
Qi et al., Mol. Plant-Microbe Interac., 24:25 (2011).
Radu et al., J. Am. Chem. Soc., 126:13216 (2004).
Ravindran et al., Nanotechnol., 16:1 (2005).
Rico et al., Agric. Food Chem., 59:3485 (2011).
Rossolini et al., Mol. Cell. Probes, 8:91 (1994)
Salata, J. Nanobiotechnol., 2:3 (2004).
Sanford et al., Methods Enzymol., 217:483 (1993).
Serag et al., ACS Nano, 5:493 (2010).
Shah et al., Biomaterials, 32:3210 (2011).
Sharma et al., Plant Mol. Biol., 51:415 (2003).
Silva et al., BMC Plant Biol., 10:291 (2010).
Slowing et al., Advanced Drug Delivery Reviews, 60:1278 (2008).
Slowing et al., J. Am. Chem. Soc., 128:14792 (2006).
Slowing et al., J. Am. Chem. Soc., 129:8845 (2007).
Slowing et al., J. Mater. Chem., 20:7924 (2010).
Solberg et al., J. Phys. Chem. B, 110:15261 (2006).
Song et al., Stud. Surf. Sci. Catal., 165:471 (2007).
Staiger et al., Curr. Biol., 4:215 (1994).
Stender et al., ACS Nano, 4:7667 (2010).
Stone et al., Proc. Natl. Acad. Sci. USA, 98:11806 (2001).
Stone et al., Wiley Interdisciplinary Reviews: Nanomedicine and Nanobiotechnology, 3:100 (2011).
Svarovsky et al., BioTechniques, 45:535 (2008).
Svarovsky et al., Mol. Pharm., 6:1927 (2009).
Thakkar et al., Nanomed. Nanotechnol. Biol. Med., 6:257 (2010).
Torney et al., Nat. Nanotechnol., 2:295 (2007).
Trewyn et al., Acc. Chem. Res., 40:846 (2007).
Trotochaud et al., Plant Cell, 11:393 (1999).
Venglat et al., Proc. Natl. Acad. Sci. USA, 99:4730 (2002).
Vivero-Escoto et al., Small, 6:1952 (2010).
Wang et al., J. Am. Chem. Soc., 132:16417 (2010).
Wang et al., J. Biol. Chem., 284:12000 (2009).
Wang et al., J. Nanosci. Nanotechnol., 11:2208 (2011).
Ward et al., Biochemistry, 21:4535 (1982).
Warzecha et al., in Genetic Modification of Plants, Springer Berlin Heidelberg, 64:23 (2010).
Wild et al., Environ. Sci. Technol., 43:5290 (2009).
Williams et al., Proc. Natl. Acad. Sci. USA, 94:10467 (1997).
Wilson, U.S. Patent Appln. No. 2011/0059529.
Wu et al., Plant Cell, 13:2841 (2001).
Wu et al., Plant Mol. Biol., 77:117 (2011).
Wymer et al., Planta, 212:692 (2001).
Xia et al., ACS Nano, 3:3273 (2009).
Yu et al., Chem. Biol., 13:723 (2006).
Yu et al., Curr. Biol., 13:179 (2003).
Yu et al., Dev., 127:1661 (2000).
Zhang et al., Phys. Med. Biol., 52:1485 (2007).
Zhu et al., J. Environ. Monit., 10:713 (2008).
Zhu ET AL., J. Phys. Chem. B, 110:10842 (2006).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A gold-plated mesoporous silicate body comprising pores and at least one agent, wherein the at least one agent is associated with the gold-plated surface or embedded in the pores.

2. The silicate body of claim 1 wherein the gold-plated surface comprises a functional group.

3. The silicate body of claim 1 wherein the at least one agent is noncovalently associated with the gold-plated surface.

4. The silicate body claim 1 which comprises two different agents.

5. The silicate body of claim 4 wherein one of the agents is in the pores and the other agent is associated with the gold-plated surface.

6. The silicate body of claim 4 wherein both agents are embedded in the pores and associated with the gold-plated surface.

7. The silicate body of claim 4 wherein one of the agents comprises nucleic acid.

8. The silicate body of claim 7 wherein the nucleic acid encodes a protein.

9. The silicate body of claim 4 wherein one of the agents comprises protein.

10. The silicate body of claim 1 wherein the at least one agent is covalently associated with the gold-plated surface.

11. The silicate body of claim 1 wherein the pores have a diameter of about 5 nm to about 50 nm.

12. A composition comprising a complex comprising a mesoporous silicate body comprising pores and at least a first agent, a calcium salt, a carrier, gold particles, and a second agent.

13. The composition of claim 12 wherein one of the agents comprises nucleic acid.

14. A method to deliver at least one agent to a eukaryotic cell, comprising:
providing a composition having the gold-plated mesoporous silicate body of claim 1; and
biolistically delivering the composition to a eukaryotic cell in an amount effective to deliver the at least one agent to the cell.

15. The method of claim 14 wherein the cell is a plant, algal, or fungal cell.

16. The method of claim 14 wherein the cell is an animal cell.

17. The method of claim 14 wherein the gold particles and the mesoporous silicate bodies in the composition form a complex.

18. The method of claim 14 wherein the at least one agent comprises DNA.

19. The method of claim 14 wherein the composition is delivered to a plant.

20. The method of claim 14 wherein the composition is delivered to cells in a tissue of an animal.
